(19) United States
(12) Patent Application Publication
(10) Pub. No.: US 2024/0221921 A1
(43) Pub. Date: Jul. 4, 2024

(54) System And Method For Integrating Digital Differential Diagnostic Examination And Medical Treatment Strategies Using Digitally Interactive Three-Dimensional, Full-Scale, Human Body Image, Modifiable Anatomical Avatars To Self-Report, Diagnose And Treat Patient Systems

(71) Applicant: Michael Hebrard, Oakland, CA (US)

(72) Inventor: Michael Hebrard, Oakland, CA (US)

(21) Appl. No.: 18/439,876

(22) Filed: Feb. 13, 2024

Related U.S. Application Data
(63) Continuation of application No. 17/126,180, filed on Dec. 18, 2020, now abandoned.

(51) Int. Cl. G16H 40/20; G16H 10/60; G16H 20/00; G16H 30/00; G16H 50/20; G16H 50/30; G16H 80/00

(52) U.S. Cl. CPC G16H 40/20; G16H 10/60; G16H 20/00; G16H 30/00; G16H 50/20; G16H 50/30; G16H 80/00

(57) ABSTRACT

An in-person physician examination scheduling and digital healthcare records management system and method for pairing patient and physician mobile applications to initiate patient communications with physicians, and for modifiable anatomical avatars to be depicted in a patient's image to self-report patient symptoms to a physician for in-person examination and treatment are disclosed. The inventive system and method provide digital communication between patients and physicians, based on patients' digital healthcare records and information obtained at in-person examinations. The inventive system and method utilize digital medical algorithms and modifiable anatomical avatars to improve diagnostic accuracy and timely treatments.

### RELATED APPLICATION

This application is a continuation-in-part of patent application Ser. No. 17/126,180, filed Dec. 18, 2020, the entire contents of which are incorporated by reference herein. This application claims the benefit under Title 35, United States Code Section 120 of the prior, copending United States application, identified above (patent application Ser. No. 17/126,180), and, insofar as the subject matter of each of the claims of this application is not disclosed in the manner provided by the first paragraph of Title 35, United States Code Section 112, Applicant acknowledges the duty to disclose to the Patent Office all information as defined in Title 37, Code of Federal Regulations Section 156(a), material to patentability as defined by Title 37, Code of Federal Regulations Section 1.56(b).

### FIELD OF INVENTION

This invention relates generally to doctor scheduling systems, and more particularly, to an in-person physician examination scheduling system and processes for pairing mobile devices to initiate patient communication with a nearby physician and for using digitally interactive, three-dimensional (“3D”), full-scale, human body image, modifiable anatomical avatars depicted in a patient's image, to self-report, diagnose and treat patient symptoms.

### BACKGROUND

Limited access to urgent medical care creates delays in treatment, increased diagnostic and treatment errors. The result increases disease severity, elevated costs, provider burn-out and a host of other problems. Indeed, the current electronic health records systems are time consuming, forcing healthcare providers to go through useless, irrelevant digital heath record tablets, slowing down providers' workflow. This problem has been an ongoing issue with existing technology, forcing physicians (due to time constraints and high patient volume) to cut and paste critical patient data in the electronic health records leading to inaccurate diagnoses and treatment efforts. Consequently, the existing healthcare system is currently wasting trillions of dollars annually, which leads to increased morbidity and mortality. Many such problems could be alleviated or eliminated with better use of existing technology. For instance, none of the existing systems include mobile applications in which there is duality in the application platform allowing a patient (using one mobile device) to initiate contact with an available physician (using another mobile device). Currently, there are no comprehensive dual mobile application devices that fully integrate with a patient's electronic health records system utilizing an interactive, full-scale, 3D anatomical avatar to provide an accurate graphic illustration of the patient's symptoms and clinical findings.

Moreover, while the scheduling of in-person, hands-on medical evaluations by board certified physicians has been and currently is being done, the current healthcare system fails to allow patients and the in-person, hands-on examining physician to utilize a digital mobile device that is integrated with the patient's electronic health record system, allowing the patient and the in-person, hands-on examining physician to gain immediate access to the patient's medical history prior to treatment. This gives the examining physician the pertinent past medial history of the patient, such as diabetes, high blood pressure, and various other medical conditions prior to the evaluation, which significantly improves the quality of delivered care in an in-office or mobile environment. The more the in-person examining physician knows about the patient's past medical history significantly increases the accuracy of diagnoses and delivery of timely treatments, improving health outcomes while lowering costs due to delayed care and/or inaccurate diagnoses.

### SUMMARY

According to a first aspect, the present disclosure provides a process to self-report symptoms to a physician for an in-person examination, comprising: transmitting a patient mobile application to a patient mobile device associated with a patient who has a medical issue and is seeking in-person examination and diagnosis of the medical issue to be treated, wherein when the patient mobile application is installed on the patient mobile device, the patient is able to seek in-person examination and treatment from an available nearby physician in a pool of nearby physicians; transmitting a physician mobile application to a physician mobile device associated with a physician who is available to examine patients nearby a physical location of the physician, wherein when the physician mobile application is installed on the physician mobile device, the identity and qualifications of the physician are verified, and upon receiving physician approval to receive nearby requests for appointments by patients, the physician is listed among a pool of physicians in an area that is nearby the physical location of the patient; receiving, from the patient mobile application running on the patient mobile device, answers to a series of questions paired with one or more 3D human anatomical models to gather initial patient data; receiving, from the patient mobile application running on the patient mobile device, an opt-in request to locate a nearby physician and schedule an in-person appointment; receiving, from the patient mobile application running on the patient mobile device, an approval to participate in and pay for an in-person examination according to the scheduling of the in-person appointment; transmitting a request to schedule the in-person appointment to the physician mobile application running on the physician mobile device; receiving an acceptance, from the physician mobile application running on the physician mobile device, to schedule the in-person appointment to examine the patient; utilizing, by the physician mobile application running on the physician mobile device, a data platform and predictive diagnoses to complete an in-person examination of the patient; and prescribing a treatment plan for the patient based on the completed in-person examination of the patient by the physician.

Patients and physicians may use the flexibility of the dual mobile application to schedule an in-person home or office visit, reducing wait times to access and deliver medical care. In addition, the disclosed invention provides a mechanism for the in-person examining physician to view on screen the patient's 3D anatomical avatar illustrating their symptoms, detailed present history of illness and/or symptoms, which correspond to the appropriate human organ system, immediate access to all pertinent health histories contained in the electronic health systems, allowing for a more accurate and timely diagnoses and treatments.

The technology in this invention pairs the human organ systems that correspond to the patient's symptoms and the physician's clinical findings. The patient is able to select or describe their symptoms utilizing a combination of a full-scale interactive 3D anatomical avatar model paired with a digitally formatted patient questionnaire to capture clinical health data for the in-person hands on examining physician to view on-screen prior to the in-person hands-on examination. Similar to the patient's use of the mobile device application, the physician performing an in-person, hands-on examination utilizes a juxtaposed image of the patient's 3D anatomical avatar model to capture pertinent clinical examination findings. Both patient's 3D anatomical avatar models appear simultaneously on the physician's mobile screen allowing the examining physician to view what the patient captured as health symptom data contrasting to the actual health physical examination data captured by the in-person, hand-on examination. The 3D anatomical avatar is in the patient's physical likeness, which improves the user experience for both patient and physician.

Illustration of the 3D full-scale anatomical human avatar model (covering more than just the head and neck) shows the patient user an exact replica of himself or herself allowing that visual display on the computer screen to represent the patient's symptoms by color coding areas of pain (i.e., red for areas that are cold, blue for areas that are numb or tingling, yellow for areas that ache or are sore). This system would accommodate a variety of different color-coding strategies to correspond to the exact specifications of the patient's symptoms. The in-depth visual display is also capable of illustrating present physical traumas, such as lacerations, skin rashes, bruising, swelling, bleeding, pus or any visual element of human injuries. The modifiable features of the 3D full-scale human anatomical avatar can also be rotated through virtual space to identify any areas of the patient's complaints such as on the soles of their feet down to the web spaces of their fingers. The zoom features of this technology give the patients a close-up view of the avatar as if they were in the physical presence of their physician examiner. In a juxtaposed fashion, the in-person, hands-on examining physician has the uploaded image of the patient's avatar anatomical model with the full illustrative depiction on the examining physician's portal screen, allowing for clinical examination findings to corroborate the patient's symptoms through clinical evaluation. The modifiable 3D full-scale human anatomical avatar model is also used by the examining physician to illustrate visually, on the physician's viewing screen, the patient's visual areas of disease or physical injuries.

For example, clinical exam findings, such as range of motion of specific areas like the shoulder, can be shown on the physician's mobile device portal. The examining physician's clinical findings are identified in their exact location using the modifiable capabilities of the 3D full-scale human anatomical avatar technology, such as skin pathologies, including discoloration (i.e., yellow for jaundice, blue for cyanosis, lacerations depicting red for bleeding, bony deformity from fractures, swelling of limbs, or abdominal bloating, warmth and coolness of extremities consistent with poor blood circulation or nerve injury). This is a limited scope of the disclosed technologies' capabilities, in which both the patient's and examining physician's illustrations of the patient's full-scale, 3D human anatomical avatar is immediately uploaded to the patient's electronic health records system to be stored, retrieved, and distributed to the appropriate healthcare agencies.

An example of the improved technologies' functionality, using patient self-reported symptoms paired with the appropriate organ systems, is as follows: a patient with a complaint of sudden back pain without any history of recent injury would typically correspond to the musculoskeletal organ system and would typically be treated with an anti-inflammatory medication. However, if the same patient over the age of 50 with a history of hypertension, alcoholism and drug abuse has a more complex health data status, which may correspond more accurately, to the vascular organ system with a possible diagnosis of an abdominal aortic aneurysm mimicking back pain. This technology coordinates human organ systems, patient symptoms and their pertinent medical histories and demographics in order to capture critical health data accurately and quickly. The current technologies lack the ease of use, flexibility and speed to evaluate, assess, diagnose and accurately treat.

The disclosed invention increases the quality of face-to-face time with the provider and patient, making the patient-physician visit more personal. Today, an increasing number of physicians are burned-out with the current system due to administrative delays caused by the current failed medical systems and medical records technology. The disclosed invention is equipped to transcribe automatically (auto-transcribe) corresponding medical reports in real-time, beginning with the patient's completion of importing health data into the system requesting in-person examination by a nearby physician. Upon completion of the in-person examination by the in-person physician, the completed medical reports are uploaded into the patient's electronic health records. The patients will retain access (24 hours per day, 7 days per week) to all of their medical records via the inventive mobile application, which includes detailed information of the physician's report, and what the patient initially reported as symptoms. The graphic illustration of the patient's 3D anatomical avatar will also be uploaded juxtaposed to the mirror image of what the physician documented as observed clinical findings during the in-person, hands-on examination by the physician.

Expanding a diagnostic and treatment library information bank is another key function of the disclosed technology apparatus. For example, when a patient's self-described symptoms are entered by the patient into the mobile device apparatus, creating a visual illustration of the patient's condition and viewed on the physician's mobile device apparatus for the purpose of performing an in-person, hands-on clinical evaluation, the examining physician modifies the patient's juxtaposed 3D, full-scale human anatomical avatar model appearing on the physician's mobile device, allowing the examining physician to view both 3D, full-scale human anatomical avatar initially modified by the self-reporting patient and the other 3D model to be utilized by the examining physician to record images comparing the physician's clinical findings to the patient's self-reported symptoms. Upon completion of the patient's evaluation by the examining physician, the system will analyze the patient's self-described symptoms (using 3D full-scale human anatomical avatar model), corresponding human organ systems and the examining physician's clinical findings to process the most accurate diagnosis and treatments based on the examining physician's knowledge, training and clinical judgment. This expanding library bank of medical information is immediately accessible to all medical physicians who utilize the system, allowing each physician user to contribute to the library bank through inputting their clinical findings for other future users to use to help diagnose and treat similar complex symptoms and diseases with more success.

The expanding library bank of medical information works through the ongoing input of clinical findings by each examining physician. The physician's clinical exam findings are captured by the system and stored with every combination of patients' self-reported symptoms that correspond to the appropriate human organ system, creating an expanding list of potential diagnoses and treatments based on best medical practices and evidence-based medicine supported by the latest medical science.

In addition, the auto-transcription system is operated by both patients' and physicians to input data, which utilizes a complex, comprehensive medical algorithm tree, that is modifiable based on the specifics of patients' medical history and current clinical symptoms, and for the in-person examining physicians' documented clinical findings, diagnosis, and treatment provided. Each answered question by the self-reporting patient's medical history and examining physician's clinical findings automatically populates into a readable format for the final transcribed medical reports.

This system does not require an independent transcriptionist, which would delay medical reporting and timely medical decisions for accurate treatments.

### BRIEF DESCRIPTION OF THE DRAWINGS

FIG. 1 illustrates an integrative diagnostic system platform according to the present invention.

FIG. 2 illustrates an integrative healthcare network according to the present invention.

FIG. 3 illustrates a Physician Mobile Application according to the present invention.

FIG. 4 illustrates a component flow diagram according to the present invention.

FIG. 5 illustrates components involved with technology security according to the present invention.

FIG. 6 illustrates a Patient Mobile Application according to the present invention.

FIG. 7 illustrates a user interface of a Patient Mobile Application according to the present invention.

FIG. 8 illustrates a user interface of a Patient Mobile Application according to the present invention.

FIG. 9 illustrates views of a Physician Mobile Application according to the present invention.

FIG. 10 illustrates a Patient Mobile Application according to the present invention.

FIG. 11 illustrates a user interface of a Patient Mobile Application according to the present invention.

FIG. 12 illustrates a user interface of a Patient Mobile Application according to the present invention.

FIG. 13 illustrates a user interface of a Physician Mobile Application according to the present invention.

FIG. 14 illustrates a user interface of a Physician Mobile Application according to the present invention.

FIG. 15 illustrates a user interface of a Physician Mobile Application according to the present invention.

FIG. 16 illustrates an integrative diagnostic database system platform according to the present invention.

FIG. 17A illustrates a Physician's user interface from a Physician Mobile Application according to the present invention.

FIG. 17B illustrates a view of a Physician Mobile Application according to the present invention.

### DESCRIPTION

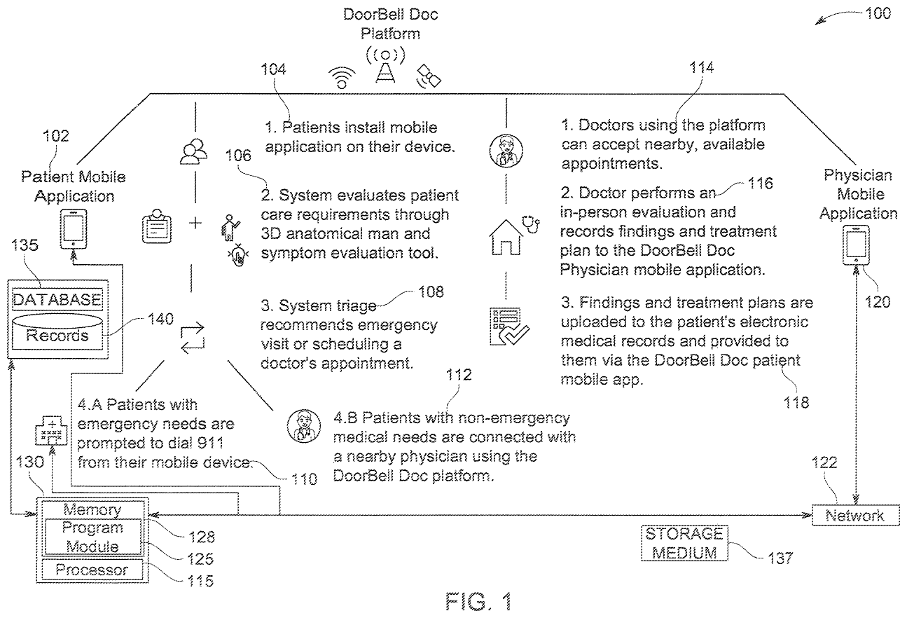

FIG. 1 illustrates an integrative diagnostic system platform, e.g., system 100 according to the present disclosure. System 100 comprises a Patient Mobile Application 102, Physician Mobile Application 120 and a means 104 for a patient-user to install Patient Mobile Application 102 on a remote device. System 100 evaluates patient care requirements utilizing a modifiable 3D full-scale human avatar in the likeness of a patient-user, to display visually on Patient Mobile Application 102 the location of a patient-user's symptomatic complaints. In addition, a patient-user may utilize a symptom evaluation tool, which is programmed to ask a patient-user pertinent medical history questions based on a patient-user's symptomatic complaints, thereby creating an automated transcribed medical history narrative that is algorithmically formatted to correlate with any of the medically relevant eleven human organ systems that are responsible for the patient-user's symptomatic complaints 106. System triage mechanism 108 allows a patient-user to activate emergency healthcare services 110 by inputting specific phrases, such as chest pain, shortness of breath, sudden loss of speech, or new onset leg or arm weakness, which will dispatch the emergency medical system to a patient-user's current location, by a mobile global positioning system (GPS) tracking device, for a patient-user to receive emergency healthcare services. System 100 further comprises a platform 112 utilizing a patient-user's Patient Mobile Application 102 for seeking non-traumatic medical treatment, recording pertinent non-emergency medical symptoms and history of illness for which a patient-user is seeking in-person, hands-on treatment by a nearby physician.

System 100 further comprises a platform 114 for a nearby physician-user to accept a patient-user's request to perform an in-person, hands-on examination utilizing Physician Mobile Application 120, which displays an on-screen juxtaposed modifiable 3D full-scale human anatomical avatar model in the likeness of the patient-user, allowing a physician-user to digitally modify a patient-user's displayed avatar pictorial image, while simultaneously viewing the initial patient-user self-modified avatar that digitally recorded the location of the patient-user's symptomatic complaints, which are self-reported by the patient-user and recorded along with a physician-user's clinical examination findings that are systematically correlated with any of the eleven human organ systems that contribute to a patient-user's symptomatic complaints, clinical diagnosis and generating a modifiable diagnosis and treatment plan 116. System 100 comprises platform 118, having the ability to upload all user-patient's electronic health record information, providing a physician-user's access to a patient-user's health history, allowing for a more detailed and precise clinical visit to facilitate rapid, efficient and high-quality level comprehensive healthcare.

For purposes of the present disclosure, network 122 comprises a group of interconnected computers. Network 122 may be classified according to a wide variety of characteristics, comprising Personal Area Network (PAN), Local Area Network (LAN), Campus Area Network (CAN), Metropolitan Area Network (MAN), Wide Area Network (WAN), Global Area Network (GAN), Internetwork (intranet, extranet, Internet) and various types of wireless networks. All networks comprise basic hardware building blocks to interconnect network nodes, such as Network Interface Cards (NICs), Bridges, Hubs, Switches and Routers. In addition, some method of connecting these building blocks is required, commonly in the form of galvanic cable (including category 5 cable). Less commonly employed are microwave links (such as IEEE) or optical cable (optical fiber).

For purposes of the present disclosure, the term computer refers to any type of computer or other device that implements software, including an individual computer, such as a personal computer, laptop computer, tablet computer, mainframe computer, microcomputer, etc. The term computer also comprises electronic devices such as an electronic scientific instrument including a spectrometer, a smartphone, an eBook reader, a cell phone, a television, a handheld electronic game console, a video game console, a compressed audio or video player such as an MP3 player, a Blu-ray player, a DVD player, etc. Additionally, the term computer comprises any type of network of computers, such as a network of computers in a business, a computer bank, the Cloud, the Internet, etc. Various processes of the present disclosure may be carried out using a computer.

Various functions of the present disclosure may be performed by one or more computers. Computer 130 comprises processor 115, memory 128, program module 125, database 135 and records 140. For purposes of the present disclosure, the term machine-readable medium comprises any tangible or non-transitory medium that can store encoding or carrying instructions for execution by a machine and that cause a machine to perform any one or more of the methodologies of the present disclosure, or that can store encoding or carrying data structures utilized by or associated with such instructions. The term machine readable medium comprises solid-state memories and optical and magnetic media. Specific examples of machine-readable media include non-volatile memory, including by way of example, semiconductor memory devices (e.g., EPROM, EEPROM and flash memory devices), magnetic disks (e.g., internal hard disks, removable disks), magneto-optical disks and CD-ROM and DVD-ROM disks. The term machine-readable medium may also comprise a single medium or multi-media (e.g., a centralized or distributed database and/or associated caches and servers) that store the one or more instructions or data structures.

Memory 128 may be implemented in a random-access memory (RAM), a hard drive, a read-only memory (ROM) or a combination thereof. One of the components of memory is program module 125. Program module 125 comprises instructions for controlling processor 115 to execute methods. For purposes of the present disclosure, the term processor refers to a device that performs the basic operations in a computer. A microprocessor is one example of processor 115.

For purposes of the present disclosure, the term database or data record refers to a structured collection of records or data that is stored in a computer system. The structure is achieved by organizing data according to a database model. The model currently in general use is the relational model. Other models (such as the hierarchical model and the network model) use a more explicit representation of relationships (see below for an explanation of various database models). A computer database relies upon software to organize data storage. This software is known as a database management system (DBMS). DBMSs are categorized according to the database model that they support. The model tends to determine the query languages that are available to access the database. A great deal of the internal engineering of a DBMS, however, is independent of the data model and is concerned with managing factors such as performance, concurrency, integrity and recovery from hardware failures. In these areas there are large differences between products.

For purposes of the present disclosure, the term data storage medium or data storage device refers to any medium or media on which data may be stored for use by a computer system. Examples of data storage media comprise floppy disks, ZIP disks, CD ROM, CD-R, CD-RW, DVD, DVD-R, memory sticks, flash memory, hard disks, solid state disks, optical disks, etc. Two or more data storage media acting similarly to a single data storage medium may be referred to as a data storage medium for purposes of the present disclosure. A data storage medium may be a part of a computer. For purposes of the present disclosure, the term data storage medium or data storage device refers to any medium on which data may be stored for use by a computer system. Examples of data storage media comprise floppy disks, ZIP disks, CD ROM, CD-R, C-RW, DVD, DVD-R, memory sticks, flash memory, hard disks, solid state disks, optical disks, etc. Two or more data storage media acting similarly to a single data storage medium may be referred to as a data storage medium for purposes of the present disclosure. A data storage medium may be part of a computer. Storage medium 137 creates records 140, including clinical data, health history with an expanding library of descriptive terms used by the patient-user for documented symptoms, medications, modifiable avatar, any of the eleven human organ systems correlating with symptomatic complaints, clinical findings, diagnostic data and treatment plans, medications and allergies.

In operation, system 100 provides a method for patient-users to directly schedule an appointment with a nearby physician-user for an in-person, hands-on physical examination. A patient-user, via Patient Mobile Application 102, creates records 140. Records 140 typically include data comprising modifiable pictorial 3D full scale human avatar images in the likeness of a patient-user, any of the 11 human organ systems correlating to signs and symptomatic complaints of a patient-user, descriptive terms to characterize and digitally display all of the patient-user's signs and symptomatic complaints (such as color changes of bleeding, infectious drainage, dislocated limb joints, swelling of skin surfaces from an anatomic region as identified by a patient-user via utilization of a modifiable 3D avatar in the likeness of the patient user). Records 140 may also comprise a patient-user's medical information from a voice-activated auto-transcription device to capture and store a patient-user's medical history narrative in a patient-user's own words, which describe the history of a patient-user's illness (such as trauma, non-trauma) and timeline of a patient-user's illness (such as within the past 24 hours, 1-3 days ago, 7 days ago, etc.). Records 140 may further comprise a patient-user's history of recent treatment for a medical condition, along with signs and symptomatic complaints that correlate to one or more of any of the 11 human organ system related to a medical condition. Records 140 may further comprise electronic health records comprising demographic and geospatial information.

Records 140 are generated by a patient-user's Patient Mobile Application 102. For example, records 140 comprising a modifiable 3D full-scale human avatar in the likeness of a patient-user is transmitted from database 135 via network 122 and displayed on a patient-user's Patient Mobile Application 102. There is no need for external picture-taking, since a modifiable 3D full-scale avatar image is already downloaded into memory 128 of system 100 and is retrievable by a patient-user. A patient-user may digitally modify a 3D full-scale human avatar image in a patient-user's likeness by orienting the image display visual characteristics of a patient-user's medical signs and symptoms (such as bleeding from any observable anatomic region, swelling of joints and limbs that are color-coded to resemble inflammation and bleeding with a reddish color for the skin, or fluid symbolizing blood flow or drainage).

Patient Mobile Application 102 transmits to processor 115, via network 122, a patient-user's modified 3D avatar image, which comprises a digitally displayed illustration of a patient-user's observable signs of symptomatic complaints along with pertinent auto-transcribed medical narrative information describing a patient-user's symptomatic complaints that correlate to any of the eleven organ systems relating to a patient-user's medical conditions and reasons for seeking an in-person hands-on examination by a nearby physician-user. A patient-user's modified 3D avatar image along with pertinent auto-transcribed medical narrative information are received and processed by the processor 115, therefore creating records 140.

According to the present invention, multiple patient-user records 140 may be produced and stored in database 135, which are representative of a patient-user's demographic profile. As database 135 of records 140 is produced, comprising a patient-user's medical history of illnesses, medical treatments diagnostic testing to determine medical causation (such as X-rays and laboratory work), mediations prescribed for a condition along with the clinical outcomes, records 140 are tracked and monitored by system 100, allowing a patient-user, physician-user and all other collaborating healthcare teams to locally and globally access records 140 for comprehensive high-quality healthcare access and delivery.

If a patient-user has emergency needs (such as shortness of breath, sudden chest pain, difficulty speaking, or sudden onset of arm or leg weakness), such a patient-user is prompted by system 100 to activate the system's 911 emergency response code via emergency response platform 110. An emergency medical system will be notified through emergency response platform 110 of a patient-user's Patient Mobile Application 102 via network 122, and provided with a patient-user's current GPS location for the provision of emergency medical services. A patient user's records 140 may be transmitted to a nearby physician-user who is connected to system 100 via Physician Mobile Application 120 for non-traumatic medical conditions via non-emergency platform 112.

A physician-user or collaborative physician-user healthcare teams (e.g., an in-person hands-on nearby medical practitioner, locally or globally connected healthcare consultants, and emergency medical facilities) may have access to records 140 via Physician Mobile Application 120 and system triage platform 108. For pertinent reference, a first record includes the patient user's symptomatic complaints illustrated by their 3D modifiable full scale huma avatar in their likeness, and the systems auto-transcribed medical history narrative that describes the history of illness in the patient owns words, and correlates to the eleven human organ systems that contribute to symptomatic medical condition(s). It is to be understood by those skilled in the that first record may be multiple past medical records stored in the patient's electronic health record profile. The in-person nearby hands-on examining physician views through the Physician Mobile application 120 the patient's first record health information and analyzes the patient's condition to either accept the patient's request for an in-person hands on examination or triage the patient to emergency medical services for a more critical level of healthcare, activating 911 to the patient's current location.

Once the nearby physician accepts the patient's request to perform an in-person hands on examination 114, the nearby physician will transmit to the patient user's mobile application device 102, via the network 122 notification of the physician's availability, credentials, and estimated time of arrival to the patient's current location to perform the in-person hands on examination. The nearby physician arrives at the patient's location and performs the in-person hands on examination, once the in-person examination is completed, the physician will input all clinical examination findings, which also includes the juxtaposed patient user's avatar image in the likeness of the patient, displaying on both avatar images, the initial patient modified 3-D pictorial digital avatar image capturing the visually observable signs and symptomatic complaints along with the second avatar image that the in-person hands physician used to record and display all pertinent observable clinical findings 116. The in-person hands on examining physician utilizes their mobile device portal 107, for viewing, analyzing and transmitting the patient's first record that includes the accompanying of the initial modified 3-D avatar in their likeness, displaying all observable clinal signs and symptoms of the patient's health conditions along with their auto-transcribed medical history narrative, as well as simultaneously transmitting the second record, the physician's documented clinical in-person hands on examination, diagnosis, and treatment plan, that is the second record, uploaded to the patient's electronic health record platform 118 via the network 122. Multiple records may be produced and received from other consultative healthcare providers and healthcare facilities, both locally and globally, transmitted via a mobile device application 102, 107 and or by healthcare facility 110 via network 122 to the database 135, and is accessible by mobile devices, 102 and 107, and healthcare facilities 110. For purposes of illustration FIG. 1 illustrates a single database, database 135. Any additional physician or healthcare facilities may access database 135 through a separate authorized mobile devices or healthcare facilities via network 122 to the patient user's electronic health record system 118.

Program module 125 may contain instructions for controlling the processor 115 to execute the functions of the computer platform, 130. Program module 125 is loaded into memory 128, and configured on a storage medium 137, which may be a floppy disc, flash drive, zip drive, etc. . . . as well random-access memory, or other type of electronic storage, located on a remote storage system and coupled to a computer 130 via network 122. Program module 125 can also contain instructions that cause processor 115 to send a medical alert to the patient about an outbreak of ongoing disease symptoms and locations of disease activity, including demographics, as well as triage the patient user's own symptomatic complaints to activate the emergency medical system, issuing a 911 dispatch protocol to the patient's real-time location. Program module creates additional records for ongoing assessment of disease progression with the patient user, and the notification of symptoms other patients living in different regions that have symptomatic conditions seeking an in-person hands on examination. The geospatial tracking method provides a patient user's record(s) and additional records of other patient users with symptomatic complaints both locally and globally. Real-time health record keeping provides local and global healthcare providers with a medically scientific source of credible health information data, previously unavailable. Now with this technology, physicians and healthcare agencies will be more effectively efficient and precise with diagnosing, triaging and treating very complex diseases as well as to monitor and prevent global pandemics by early recognition of health threats. The tracking of symptomatic conditions, diagnosis and treatments may be stored in the patient's electronic record.

Program module 125 can also contain instructions that cause the processor 115 to digitally create the modifiable 3D human anatomical model (a CGI representation of a 3D human anatomical model on a computing device screen) to help patients explain their symptoms and improve the provider's understanding of the patient's complaints and guide the provider's evaluation diagnosis and treatment plan. The digitally modified avatar may display various disease conditions with skin color changes such as jaundice yellowish skin, related to liver diseases. The modifiable 3-D avatar in the likeness of the patient user may also display but not limited to broken bones, joint dislocations, bleeding cuts, bruising and swelling on any visible part of the human body, by manipulating the digital avatar 360 degree rotationally on the mobile device portal screen. Additionally, the digital anatomical image may be enlarged and or enhanced to more specific and detailed anatomical locations, for the precise display of the patient's symptomatic characteristics, and for the in-person hands examining physician documented clinical examination findings. Program module creates 2 separate but identical digitally modifiable 3-D full scale human avatars in the likeness of the patient user. The first digital avatar image appears on the patient mobile device screen and used by the patient to illustrate symptomatic complains, which will assist the examining physician in better understanding the patient's complaints and helps the in person examining physician to conduct a more clinically precise examination to enhance diagnostic accuracy and examination efficiency. Only the second avatar appears juxtaposed fashion to the patient's modified avatar on the physician mobile application screen, allowing the in-person hands physician to compare both the patient's symptomatic avatar to their clinically documented avatar, for more diagnostic accuracy and appropriate treatment 116 and 118.

System 100 provides an effective way to store records that chart an entire sequence of patients' symptomatic complaints utilizing digitally modifiable 3-D full scale human avatars in the likeness of the patient users and are correlated with 11 human organ systems, with a corroborated accurate diagnosis and treatment provided by a nearby in-person hands on physician. This sequence of access to comprehensive high-quality healthcare enables both patients seeking in-person hands on care and in the-person examining physician, to form a local and global network of healthcare providers for the collaboration of effective and timely healthcare, which will save lives and healthcare spending. With the satellite internet connection, remote areas where healthcare was lacking can now be accessed and delivered quickly.

System 100 also allows a patient to directly schedule and interact (without a middle person) with a nearby physician for an in-person hands on clinical examination. This reduces waiting times for appointments, which may be months or days, and the patient is more empowered to seek medical help sooner rather than later.

System 100 also allows an in-person nearby physician to coordinate with patient's primary care physician for ongoing continuity of healthcare and the patient is now participating more actively in their own healthcare. This system allows for the use mobile application devices to assist healthcare providers to establish a protocol of accountability by following up on documented patient health records and the system's timed interval alerts prompting healthcare providers' follow up with the patient and other pertinent healthcare team members to collaborate with their portion of the patient's treatment, while keeping the patients apprised of the treatment recommendations and results. The growing comprehensive healthcare team can all find access to the patient's electronic health records via network, shared amongst medical professionals who are responsible for the patient's treatment, such as general practitioners, other medical specialists, surgeons, pharmacist and social workers.

System 100 further provides memory 128 in communication with the processor 115. Machine-readable instructions in program module 125 are resident in memory 128, for execution by the processor 115 to cause the processor to carry out the steps of the methods described herein. Instructions 125 are stored on a storage medium 137 and in turn may be physically distant to the processor 115, local processor 115, or some combination of local and distant.

Processor 115 is further in communication with a database 135 for storing a record, e.g. . . . , record 140. It is to be understood that multiple records may be stored in database 135. The record may include the data record 140 described above or, alternatively the record may include physician data.

For example, the record may include physician data such as treatment data, location data and published medical articles which cover all pertinent medical conditions for the physician's reference. The location data is the location is a location of the physician or a physician's office and includes a latitude/longitude (lat/lon) coordinate 112. By this method, a patient user may access the record in the database 140 via the patient mobile application 102 and the processor 115 to obtain the location of the nearby physician available to meet them at their remote location or nearby physician's office. This will assist the patient in accessing the physician who is able to perform the in-person hands on examination and to recommend further treatment services based on the patient's medical needs. In addition, the patient user may access the data to select a specific physician specialist based upon experience data 114.

System 100 also creates geospatial monitor tracking of disease activity locally, regionally and internationally. The method of machine learning enhances this diagnostic and treatment platform process by utilizing the 11 human organ systems to scientifically correlate all symptomatic complaints with the specific organ system or systems responsible for their clinical expression and the in-person hands clinical examination findings correlation with the 11 human organ systems, producing a more accurate diagnosis and appropriate treatment 116 and 118.

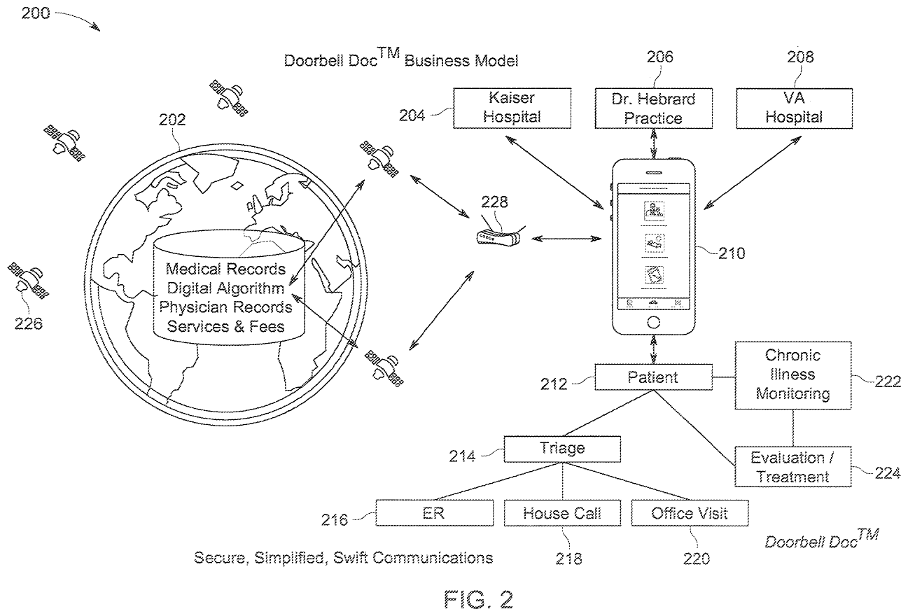

FIG. 2 illustrates an integrative healthcare network, Kaiser Hospital facility 204, medical physician 206, and Veterans Hospital facility 208 allowing the patient's self-selection using the patient mobile application device 210 directly accessing a physician's mobile application device 210 directly scheduling medical examination appointment with a nearby physician on a physician mobile application device 210, or patient seeking treatment at healthcare facilities 204 and 208, medical evaluation system with local and global access for diagnosis and treatment using earth orbiting satellites 226 satellite connective access utilizing wireless satellite routers 228, mobile device instrument 210 utilizing a digital algorithm to triage 214 patients for emergency room visits 216, house calls 218, office visits 220, monitoring patient's chronic disease 222. For the purposes of the illustration only one mobile application is shown which is representative of the patient app and the physician app.

System 200 provides medical records, digital algorithm, physician records, services and fees, 202. System 200 provides healthcare access to healthcare facilities 204 and 208. Record 202 is also in communication with orbiting earth satellites that are connected to wireless routers 228, providing GPS tracking globally without the limitations of cellular internet that is are land based cables and are not able to go specific geographic locations such as the satellite transmissions. Healthcare information can be shared with other medical providers anywhere on the globe instantaneously. This allows the patient to travel all over the world and the healthcare information goes with them with accessibility for the patient and other potential examining physician. Patients can digitally pay for healthcare services on the spot. The global capacity of this technology's system can be translated in any language for the convenience of the patients and the physicians providing care.

System 200 illustrates the technology platform's capacity to increase and enhance its memory bank of diagnosis and treatments based on the combination of symptoms and clinical examination findings correlating to the 11 human organ systems. The technology that is rooted or created out from a medical science platform which allows the system to have endless iterations of diagnostic capacities to comprehensive diagnose and treat. The digital pictorial images are already created within the framework of technology's platform program module 125 and external pictures don't need to be taken to verify the presence of observable disease conditions. The modifiable 3-D full scale human avatar pictorial image in the likeness of the user is the virtual representation of the patient user allowing the physician to remotely observe the patient's observable symptomatic complaints before the in-person hands on examination.

System 200 illustrates Patient's mobile device, the globally integrated database system which is a record storage database 202 that includes a modifiable 3-D full scale human avatar image in-likeness of patient user, patient's medical records, library bank of various descriptive symptomatic complaints that medically correspond to the 11 human organ systems which are contributing to the expression of patient's clinical symptoms such as headaches. For example, symptomatic headache complaints may correspond to the neurological organ system's, the 5th cranial nerve, which is trigeminal nerve, the largest of the cranial nerves and provides sensory and motor innervation to the head and face, and pain is usually presents as symptomatic complaints involving the cheek, jaw, teeth, gums, lips, and the eye and forehead. The various combinations of patients' symptomatic complaints are stored in the record database system, and with all patient user's symptomatic complaints are uploaded in the platform, exponentially expanding the record database, while continuing the correlation of the programmed medical history narrative algorithms with the appropriate 11 human organ systems that are responsible for their symptomatic complaints and final clinical diagnosis and treatment plans. After each finalized patient encounter, their demographics such as age, gender, ethnicity, prior and current health conditions are uploaded into the record database system, allowing for geospatial monitoring locally or globally of symptomatic conditions and disease activity. With every patient user's input into the record database platform, the patient's various symptomatic complaints, enhances the system's ability and correlation capacity for more clinical assessments and diagnostic accuracy along with more timely comprehensive healthcare delivery.

The database system utilizes a medical triage database evaluation system, as when symptomatic complaints are involving uncontrollable bleeding, sudden onset difficulty speaking or movement of arm or leg, shortness of breath or chest pain, the database system automatically activates 911, the emergency medical system to the patient's identified location, this database stores a chronic illness monitoring platform system 222 for communications among patients, healthcare network systems, including but not limited to medical physicians and collaborating connection with healthcare facilities around the world via satellite internet.

System 200 embodiments illustrate the following with some illustrations not captured in this drawing but located in other drawings herein, including but not limited to globally accessible patient record database system, patient's past medical history, network of healthcare facilities, orbiting satellites for, earth-based routers, nearby physician traveling by car for in-person hands on examination, record database system that includes juxtaposed modifiable 3-D full scale human avatar image in-likeness of patient user, allowing each physician user to document their observable clinical examination findings in conjunction with the utilization of the programmed examination template that correlates to the specific organ system or systems that have been verified through the database system as appropriate for physician's use of their documented clinical findings or allow the examining physician's clinical judgement to edit or modify the exam documentation format, which correlates most accurately with patient's symptomatic complaints. Upon the in-person hands on physician's clinical decision to utilize the specific clinically programmed examine documentation template, the database system customizes the appropriate clinical examination format for the examining physician to record all their examination findings for the most accurate diagnosis and treatment plan. This system enhances the examining physician's ability to methodically and efficiently evaluate every patient's symptomatic complaint with more medically scientific precision. Over the course with every patent and physician's input, all clinical health data is uploaded into the patient's medical records, with exponential expansion of the library bank storage of various symptomatic complaints, diagnosis and treatments, the 11 human organ systems corresponding to the various symptom complaints and clinical examination findings that mathematically utilizes the programmed algorithmic templates to find precise diagnoses and treatment plans which may be modified by the examining physician.

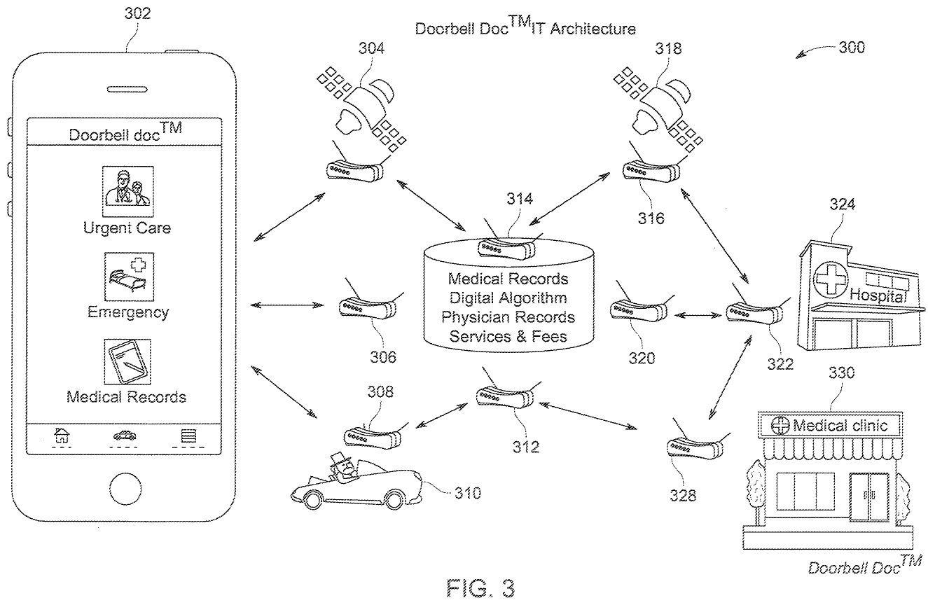

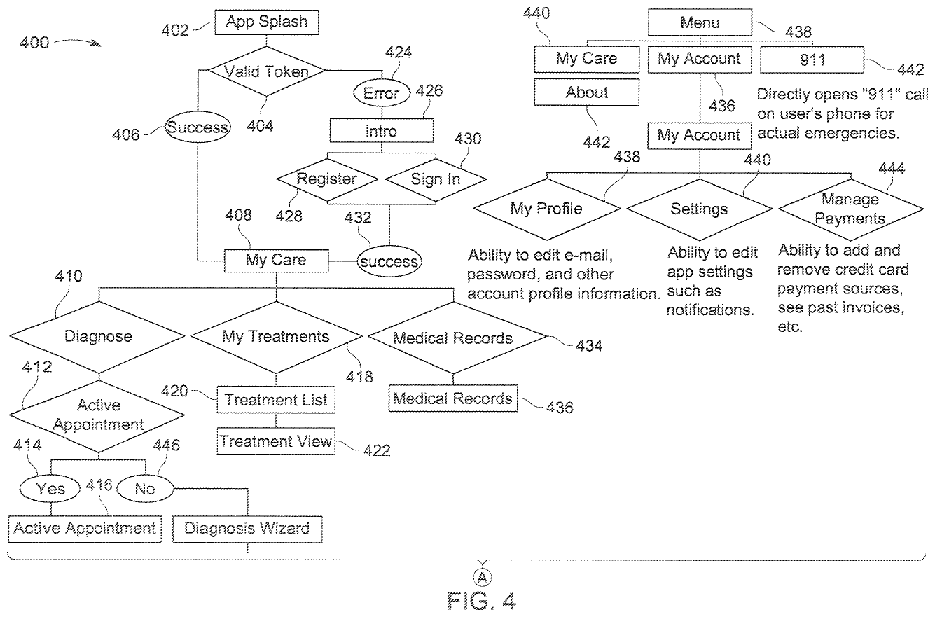

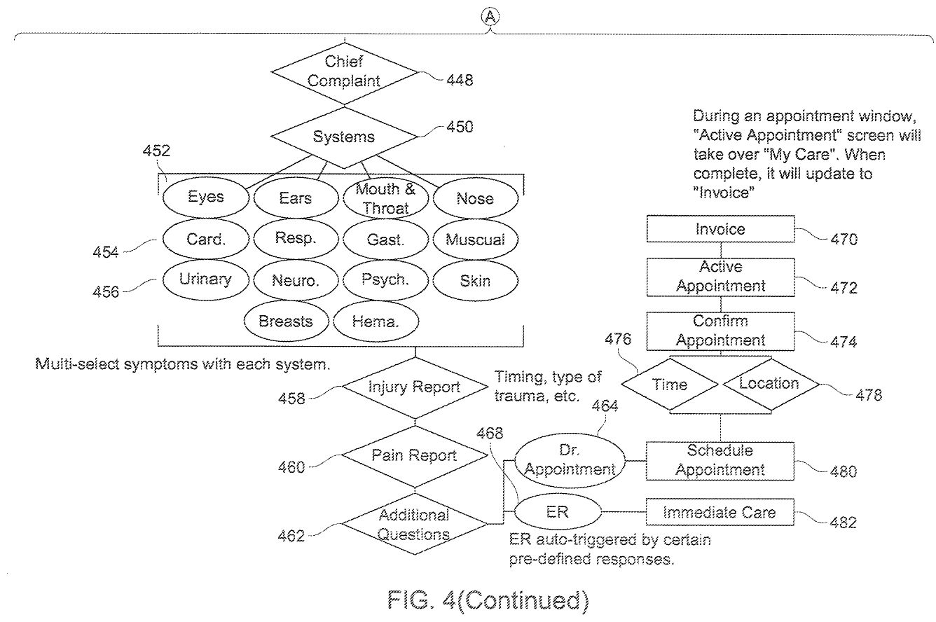

FIG. 3 system 300 illustrates Physician's Mobile Application Device 302, displaying patient's digitally modifiable avatar image, triage for services needed, view of electronic medical records, receiving and transmitting patient's health record information, digital algorithm for the symptomatic complaints and clinical examination findings that correlate to the 11 human organ systems responsible for disease symptoms and diagnosis, voice and touch activated auto-transcription, are retrieved from and stored in database 314, shared healthcare information among patients, physicians, and healthcare facilities 324, 330 via wireless routers 306, 308 and 316, to orbiting satellites 304, 318. System 300 provides an illustration of physician driving to the patient's physical location to perform in-person hands on physical examination 310. System 300 further provides a method for using a series of programmed medical algorithms that correlate every patent's symptomatic complaint and clinical examination findings to 11 human organ systems that are responsible for their clinical symptoms' manifestation and clinical diagnosis. The programmed medical algorithms, provides the patient user with a method for answering pertinent medical history questions that correlate their symptoms to the 11 human organ systems that may be responsible for the patient's symptoms and clinical examination findings. Therefore, the programmed medical digital algorithm sequencing creates a clinical examination platform for a more efficient and precise diagnostic accuracy and treatment plan, stored in the record database 314.

System 300 utilizes the physician's mobile application device 302 to communicate with a database 314 for storing a physician record, e.g., record 140. It is to be understood that multiple records may be stored in database 314. One or more healthcare facilities 324 and 330 may be in communication with the mobile physician 310, via the mobile device 302, by transmitting and receiving via wireless routers 306, 316, and 322 to earth orbiting satellites 304 318 records from the database 314, via the network 122.

FIG. system 400 is a component flow diagram, a method for illustrating the patient user's interface Application Flow Chart for the Patient's Mobile Application Portal device to schedule an in-person hands on examination by a nearby physician, record and display all symptomatic illnesses into the patient's database system utilizing the integration of the 11 human organ systems to precisely report history of clinical conditions and reasons for seeking in-person hands on examination by a nearby physician, the technology platform database allowing the patient user to sign-in or register for the App, edit and update their payment account information, health history record information, activate the 911 emergency medical system for their emergency healthcare needs.

System 400 at step 402 is an application splash which for our purposes is the first graphical notification the patient user receives when they visit the app 402. This screen will illustrate the apps digital logo “Welcome to Doorbell Doc” . . . . At step 404 is token-based process allowing the patient user access to an online account by entering a code sent as a one-time password, using a fingerprint to unlock a mobile phone, and accessing the platform system through their mobile application device, computer, or laptop 404. At step 406, the patient user has successfully accessed the platform. At step 408, the patient the portal screen will display ‘My Care” prompting the patient to select menu options for their purposes such as diagnoses for current health conditions 410 leading to next step to activate an appointment for scheduling with a nearby physician and or physician office or confirming a previously scheduled appointment 412.

At step 414, the patient has an active appointment, and the patient user confirmed previously scheduled appointment 416. At steps 418, the patient may use the patient's mobile app device to view their past medical records 434 and 436, including but not limited to their symptoms, diagnosis, name and specialties of physicians seen and their recommendations, results of past diagnostic testing such as chest X-rays, but not limited to blood work and medical specialty referrals, and access to physician's records pertaining to the patient's treatment of their symptoms 420, including their diagnosis, and treatments provided by date in chronological order. At step 438, “My Profile” demographics which includes age, ethnicity, gender, occupation, disabilities, address, this step allows the patient to edit password authentication key . . . the menu selection appears on the patient's mobile app device after successful access to the system via valid token 404. At step 440, “My Care” appears on the patient mobile app screen, along with menu selection choices of “My Account” 436, and “911”, 442 where the patient user can access 911 through their portal device which gives local emergency medical services and medical facilities immediate access to the patient's records 140 stored in the database 135.

System 400 illustrates in step 436 “My Account” the patient's access to view and edit personal information 440, including but not limited to health data information, changes in address, and billing information 444, etc. At step 442, this is like steps 410, 418, and 434. At step 446, the patient has no previously scheduled appointment, and they seek to schedule an appointment to get a diagnosis and treatment 446. At step 448, the system quires the patient for their chief complaint(s) and or reasons for seeking an in-person hands on physician examination by a nearby physician. At step 450, the patient has already entered the chief complaint or symptomatic complaints into the system via patient mobile application portal device, and now the system correlates the patient symptomatic complaints with the appropriate 11 human organ systems 452, 454, and 456, responsible for their symptoms and clinical diagnosis. At step 458, the patient is queried by the sequencing of the programmed medical algorithm that customizes questions involving the history and potential causes of the patient's current medical condition(s) and their symptoms that correlate to the 11 human organ systems, including but not limited to trauma vs non-trauma, the time of onset, length of symptoms, severity, location of symptoms, and any treatments provided, either self-procured or professionally rendered.

At step 460, the character of pain if present as part of the patient's symptoms is also queried by the sequencing of the programmed medical algorithm that correlates the various 11 human systems that may be responsible for the patient's pain. For example, low back pain may be caused by kidney stones and not lumbar muscle strain as part of the differential diagnosis, which is expeditiously solved through the technology's digital medical algorithm that enhances the examining physicians' diagnostic accuracy and treatment plan. The system can query additional questions 462 based on the potential 11 human organ systems 452, 454, and 456 involved in the patient's symptomatic complaints, such as headaches, fever, chills, nausea, vomiting, diarrhea, chest pain, muscle aches and coughing up sputum. This complex array of medical signs and symptoms are selectively analyzed by the processor 115 and the patient may retrieve their previous medical records record 140 stored in the database 135, including but not limited to the patient's past medical history of similar conditions or similar conditions experienced by other patients with similar complaints, are retrievable by the patient's mobile app device and the physician's mobile app device via the network 122. At step 464, the patient has reached the nearby physician's mobile app device via the patient's mobile app device, to schedule an appointment for in-person hands on examination. At step 480 the patient has scheduled an appointment with an in-person hands-on examination by a nearby physician. At step 476, the time of the scheduled appointment is proved to the patient by the nearby physician providing the in-person hands on examination. At step 478, the location of the in-person examination by a nearby physician is confirmed. At step 474, time and location are simultaneously confirmed by both patient and physician. At step 472, the scheduled appointment is uploaded into the system into the patient's record 140, stored in database 135. At step 470, during an appointment window, “Active Appointment” screen will over “My Care”. When this process is completed, it will update to “invoice. At step 468, illustrates the emergency medical systems activation by patient input of key words in the system such as but not limited to chest pain, shortness of breath, uncontrollable bleeding, and sudden onset of weakness in the arm or leg or sudden difficulty speaking, 911 is dispatched to patient's current location for immediate care 482, via the patient's mobile portal appl device via the network 122. Referring to step 424, the patient was unsuccessful in authentication process as the code was entered incorrectly and needs to be re-entered 430 or they need to properly register 428, then successful access is achieved 432. The patient's next step begins at step 408 and the patient's mobile app flow chart will continue in programmed sequence.

Physicians Mobile Application Flow Chart. System 400 also shows component flow diagram, a method for illustrating the physician user's interface Application Flow Chart for the Physician's Mobile Application Portal device to perform an in-person hands on examination on a patient seeking an in-person examination by a nearby physician, record and display all observable clinical examination findings, accessing the database system for pertinent medical records, utilizing the integration of the 11 human organ systems to assist the guiding the physician to perform a precise and efficient in-person hands clinical examination on a patient seeking an in-person examination.

System 400 at step 464 illustrates Dr. Appointment, is an application splash which for our purposes is the first graphical notification the physician user receives when they visit the app 464. This screen will illustrate the apps digital logo “Welcome to Doorbell Doc” . . . you have a request for in-person hands on examination by a nearby patient seeking medical treatment”. Like step 404 is token-based process allowing the physician user access to an online Physician Mobile App portal device, by entering a code sent as a one-time password, using a fingerprint to unlock a mobile phone, and accessing the platform system through their mobile application device, computer, or laptop like 404. Like step 406, the physician user has successfully accessed the platform. Like step 408, the physician's mobile app portal screen will display the patient's chief complaint(s) accompanied by the patient's digitally modifiable 3-D full scale human avatar in the likeness of the patient, illustrating the exact anatomical location of the signs and symptoms of the patient's condition. The physician also views the patient's medical history narrative programmed by the digital algorithm created by the program module 125 and stored in the database 135, and auto transcribed by the processor 115 under the instructions from the program module 125.

Like step 414, the physician confirms their availability for the active appointment, and the patient user also confirms their willingness for the scheduled appointment like 416.

Like steps 418, the physician may use the physician's mobile app device to view the past medical records 434 and 436, including but not limited to their symptoms, diagnosis, name and specialties of physicians seen and their recommendations, results of past diagnostic testing such as chest X-rays, but not limited to blood work and medical specialty referrals, and access to physician's records pertaining to the patient's treatment of their symptoms like 420, including their diagnosis, and treatments provided by date in chronological order. Like step 442 the physician user can access 911 through their portal device which gives local emergency medical services and medical facilities immediate access to the patient's records 140 stored in database 135. System 400 illustrates like in step 436 “My Account” the physician can access to view and edit their professional information like 440, including but not limited to specialty training information, changes in address, like 444, etc.

Like step 464, the patient's request for an in-person hands examination has reached the nearby physician's mobile app device via the patient's mobile app device, to schedule an appointment for in-person hands on examination. Like step 476, the time of the scheduled appointment is approved by the patient and by the nearby physician providing the in-person hands on examination. Like step 478, the location of the in-person examination by a nearby physician is confirmed. Like step 474, time and location are simultaneously confirmed by both patient and physician. Like step 472, the scheduled appointment is uploaded into the system into the patient's record 140, stored in database 135.

System 400, in the following flow chart illustration while some steps are not captured in this drawing but located in other drawings herein, including but not limited to globally accessible patient record database system, patient's past medical history, network of healthcare facilities, orbiting satellites for, earth-based routers, nearby physician traveling by car for in-person hands on examination, record database system that includes juxtaposed modifiable 3-D full scale human avatar image in-likeness of patient user, allowing each physician user to document their observable clinical examination findings in conjunction with the utilization of the programmed examination template that correlates to the specific organ system or systems that have been verified through the database system as appropriate for physician's use of their documented clinical findings or allow the examining physician's clinical judgement to edit or modify the exam documentation format, which correlates most accurately with patient's symptomatic complaints.

System 400, the step for the physician's in-person hands on examination, the physician can utilize the specific clinically programmed digital algorithm template, in which the program module 125 created the most appropriate clinical examination format based on the patient's symptoms and correlation with the 11 human organ systems that may be responsible for the patient's symptoms and clinical diagnosis. This process assists the in-person examining physician's ability to perform a more focused and efficient medical examination. System 400 utilizes the system's machine learning platform, that matches the patient's symptoms and the physician's documented examination findings, generating a list of the most medically probable diagnosis and treatments based on the latest medical science and standard of care, allowing the examining physician to select from the programmed diagnosis and treatments or customize their own based on their medical knowledge and years of clinical experience.

System 400 enhances the examining physician's ability to methodically and efficiently evaluate every patient's symptomatic complaint with more medically scientific precision. System 400 stores in its memory storage 137 every patient's symptomatic complaint and physician's clinical examination findings, all programmed generated diagnosis and treatment plans, with the outcomes of all treatments are stored and retrievable by the system database 135.

System 400, all clinical health data is uploaded into the patient's medical records, with exponential expansion of the library bank storage of various symptomatic complaints, diagnosis and treatments, the 11 human organ systems corresponding to the various symptom complaints and clinical examination findings that mathematically utilizes the programmed algorithmic templates to find precise diagnoses and treatment plans which may be modified by the examining physician. All stored diagnoses may be retrieved by other examining physicians in the future. This system will continue to learn and learn based on the user's input from patients and physicians.

System 400 illustrates embodiments illustrate the present disclosure which has been described with reference to one or more exemplary embodiments, it will be understood by those skilled in the art that various changes may be made, and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure is not limited to the embodiment(s) disclosed as the best mode contemplated, but that the disclosure will include all embodiments falling within the scope of the appended claims.

At step 468, illustrates the emergency medical systems activation by patient input of key words in the system such as but not limited to chest pain, shortness of breath, uncontrollable bleeding, and sudden onset of weakness in the arm or leg or sudden difficulty speaking, 911 is dispatched to patient's current location for immediate care 482, via the patient's mobile portal appl device via the network 122. At step 424, the patient was unsuccessful in authentication process as the code was entered incorrectly and needs to be re-entered 430 or they need to properly register 428, then successful access is achieved 432. The patient's next step begins at step 408 and the patient's mobile app flow chart will continue in programmed sequence.

System 400; Physician Mobile Application. System 400 illustrates the physician mobile app flow chart but for our purposes, the platform mechanism that utilizes the two separate mobile device instruments that display an on screen 3-D full scale human anatomical avatar model in the likeness of the user, which is a digitally modifiable pictorial image that records and displays the patient's symptomatic complaints and physician's clinical examination findings, utilizing the in-person hands on examining physician's knowledge for the integration of the 11 human organ systems that contribute to patient's symptomatic complaints, and clinical diagnosis. FIG. 1 includes the technology platform's ability to upload all the patient's electronic health record information, providing the examining physician access to the patient's pertinent health history, allowing for a more detailed and precise clinical visit, to facilitate rapid, efficient, and high-quality level comprehensive healthcare. The breath of the technology platform invention is capable of triaging a patient's symptomatic complaints, such as short of breath, which would dispatch 911 to the patient's location.

On the patient's mobile device portal screen, there is modifiable 3-D Human Anatomical Avatar digital Model, in the likeness of patient user, allowing each patient using their mobile portal device instrument, to manipulate real time their anatomical pictorial digital image in their likeness, recording and displaying on their mobile device portal screen, all of their observable symptomatic complaints, and sending that illustrated digital image along with their medical history narrative, reasons for seeking an in-person hands on examination, to a nearby physician's mobile portal device. Once the nearby examining physician receives the patient's request for an in-person hands-on examination, the physician's mobile portal device screen will display the patient's recorded avatar digital image showing the precise anatomical locations of the patient's visually observable symptomatic complaints, highlighted by digital images depicting skin color changes, swelling on the skin's surface, deformities of joints, and fluid discharge from various anatomical regions, not excluding bleeding or pus from infected body parts, etc. . . . . All visually observable symptomatic health conditions can be displayed on the patient's mobile portal device screen instrument, allowing the patient to modify or manipulate real time their 3D pictorial image in their likeness, to precisely illustrate the location and character of the current physical health that correspond to one or more of the 11 human organ systems.

On the nearby physician's mobile device portal screen instrument, the patient's symptomatic modified digital image is displayed alongside an exact juxtaposed modifiable pictorial digital image, (which doesn't have the patient's displayed modified symptomatic illustrations), allowing the in-person hands on examining physician to modify the juxtaposed pictorial image in to record and display their clinical examination findings. Once the physician completes their in-person hands-on examination, the physician records their clinical examination findings, displayed on the juxtaposed avatar image, which is alongside the patient's initial digital avatar image, that recorded and digitally displayed all the visually observable symptomatic complaints and their precise anatomical locations.

With the two side by side visually modified pictorial digital images in the patient's likeness, each examining physician is able to compare the patients digitally illustrated symptomatic complaints with the physician's recorded modifiable pictorial digital images of their clinical exam findings. This process provides every examining physician a more precise clinical examination instrument tool which utilizes the 11 human organ systems, as it compares the patient's symptomatic complaints in conjunction with physician's clinical examination findings, allowing for a more precise, efficient, and quicker diagnostic decision making for the treatment of patients, while utilizing the physician's medical knowledge of the 11 human organ systems' contribution to medical diseases.

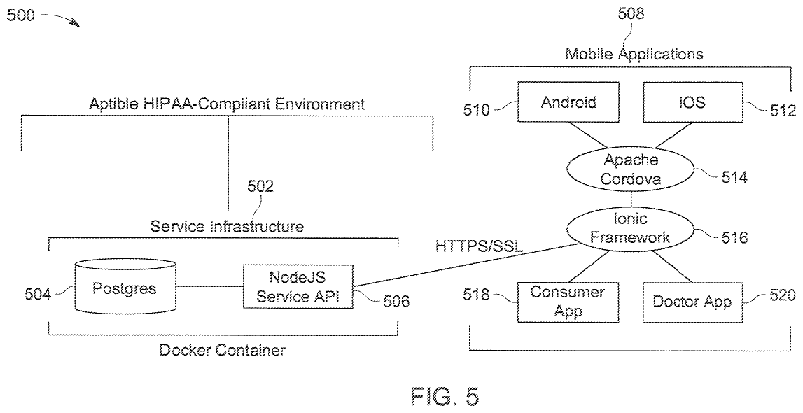

FIG. 5 system 500 illustrates the components involved with the security of the technology platform system diagnostic database including the service infrastructure 502. HIPPA-Compliant Environment 502 automates and enforces all needed infrastructure security and compliance controls required to safely store or process HIPPA-protected health information. HIPPA compliance for our purposes means adherence to the physical, administrative, and technical safeguards outlined in HIPPA, which is the right to privacy, security, and notification when compromised in a breach. System 500 provides an illustration of the PostgreSQL infrastructure 504 which is an object-oriented relational database management system to store data securely for retrieval at the request of the software application, processor 115.

System 500 illustrates NodeJS service (API) 506 and for our purposes, JavaScript runs outside the browser-on the server. It is used for building single-page applications and dynamic websites due to its ability to handle multiple requests asynchronously. It serves content to users, improving the overall user experience, allowing the free exchange of data. NodeJS can handle many simultaneous connections through network 122, messages are delivered between the patient's mobile app and physician's mobile app devices via the network 122, transmitting over the cellular or satellite internet channels, with maximum rate of speed.

System 500 further illustrates various mobile devices 508, Android 510, IOS on Apple 512, and 514 is discontinued and replaced by Ionic framework 516. System 500 further illustrates Ionic framework and for our purposes Ionic framework 516 empowers the capacity of cross-platform mobile apps to work together on the same system platform using HTML, CSS, and JavaScript which can deploy a native iOS or Android app, native desktop, or web app, all from a single shared code. System 500 further illustrates patient mobile app (Consumer App) 518 and physician mobile app (Doctor App) 520.

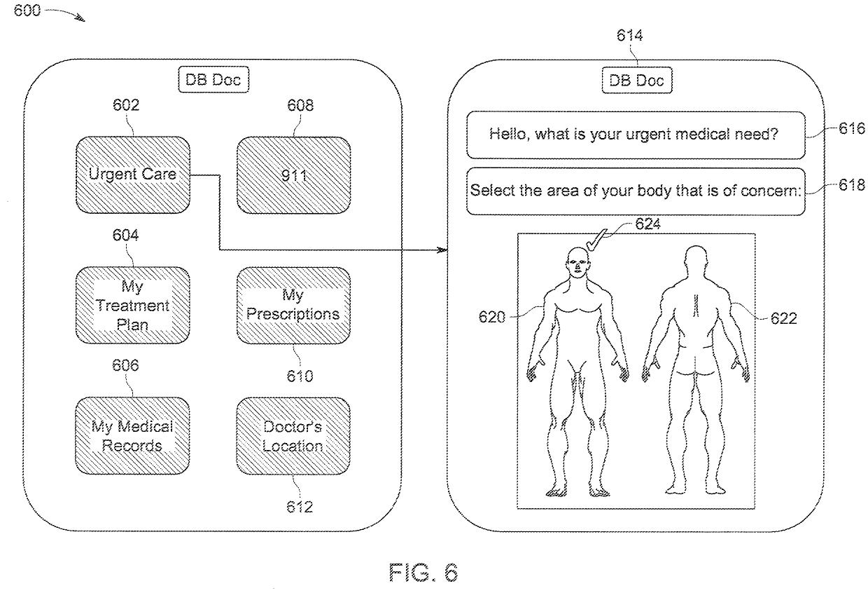

FIG. 6 system 600 is an illustrates the views of the patient user's interface of Patient's Mobile App portal screen device, displaying menu selection options, including Urgent Care 602, My Treatment Plan 604, My Medical Records 606, 911 dispatch 608, My Prescription, Doctor's Location 612. System 600 illustrates by showing the user interface of the patient's mobile app screen displaying querying of the patient regarding the need for scheduling in-person hands on examination by a nearby physician 616 and the selection for the anatomical region of symptomatic concern 618.

System 600 illustrates a patient's mobile app screen displaying a digitally modifiable 3-D full scale human avatar pictorial image 624 side by side views of the front view 620 rear view 622. System 600 illustrates a method and shows the user interface of the patient's mobile app device to utilize the modifiable 3-D full scale human anatomical avatar in the likeness of the patient user. System 600 shows user interface 620 and 622, that is displaying a digital modifiable 3-D full scale human avatar that the user can rotate and modify on the mobile device portal screen, the digital 3-D image to locate the precise anatomical areas of symptomatic complaints, such as the head 624, with capacity to rotate, enhance or enlarge the digital avatar's image to emphasis the exact anatomical location of the patient's symptomatic complaints. The medical algorithm platform allows the retrieval from its programmed library bank database 314, all pertinent visually observable human pathological signs and symptomatic conditions, including but not limited to bleeding and or swelling from the head 624, displaying the exact anatomical location as identified by the patient user on their portal screen device, an actual visual pictorial display of their symptomatic condition. 928. For our purposes the digitally modifiable 3D full-scale human pictorial image involves using software to create a three-dimensional model of the patient user, a digitally modifiable 3D full scale image in the likeness of the patient user.

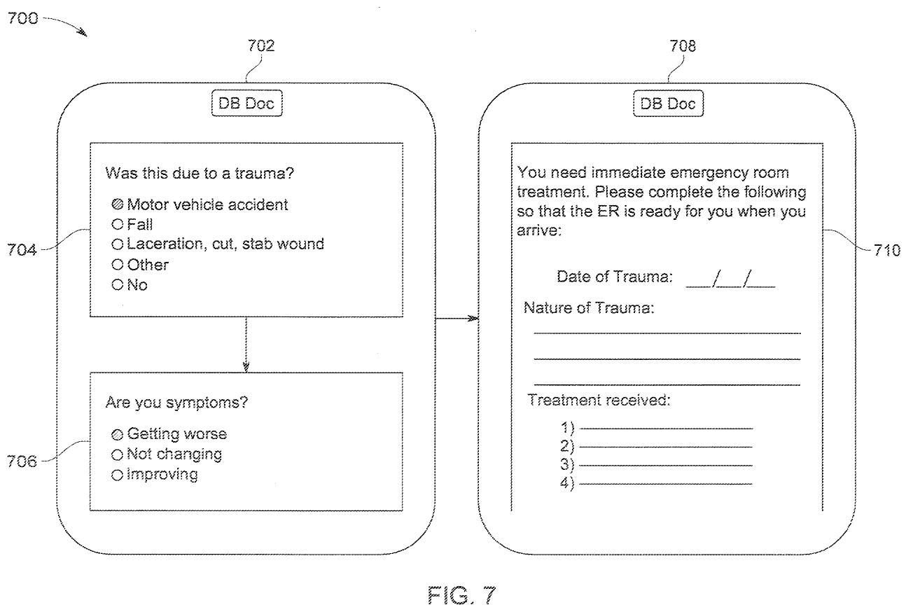

FIG. 7 system 700 shows user interface 702 and 708 a method platform utilizing the patient's mobile device instrument for seeking emergency medical treatment due to trauma 704 recording pertinent worsening medical symptoms 706 and history of illness that the patient is seeking in-person hands on treatment by a nearby physician. System 700 illustrates and shows the user interface the medical algorithm querying the patient user about the need for emergency medical services 710.

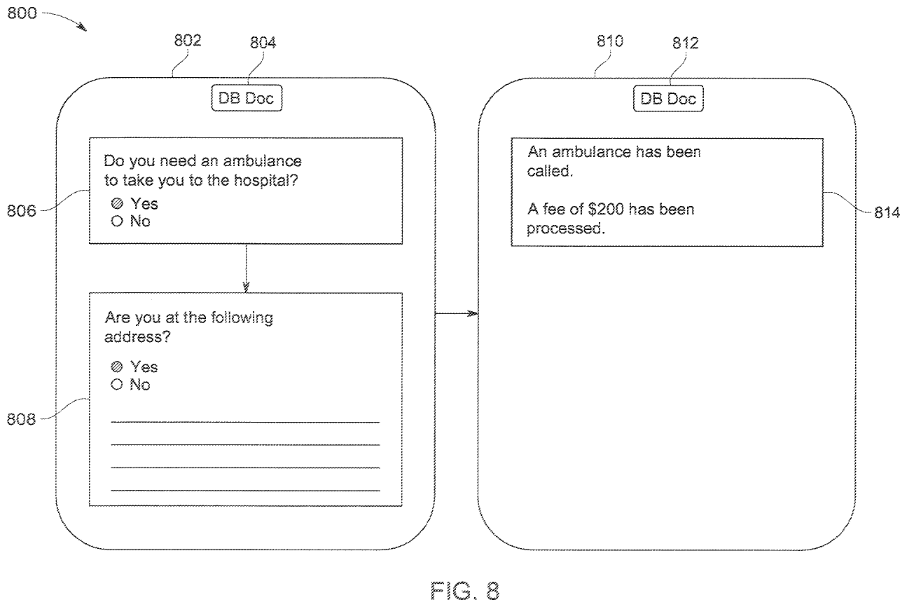

FIG. 8 system 800 displays the patient's mobile interface 802 and 810, continues showing the user interface on the patient's mobile app screen the Doorbell Doc app logo 804 and 812 from FIG. 7 system 700. The digital algorithm platform queries the patient user regarding transportation for emergency services, 806 and 808 confirming the address for dispatching 911. 911 is dispatched from the patient's mobile app device by GPS location of the patient along with the patient's confirmation of their exact location and fee for service payment processing automatic charge for the Doorbell Doc service 804.

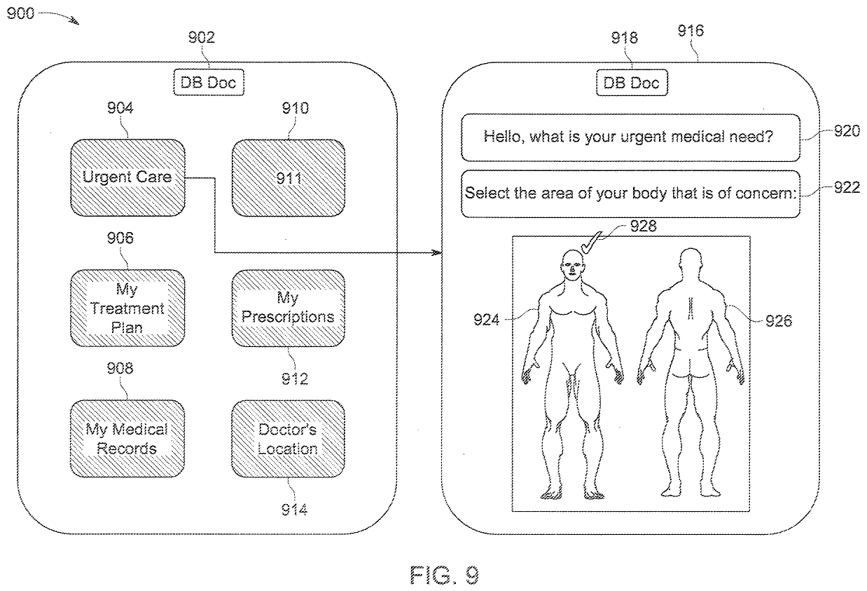

FIG. 9 system 900 is an illustrates the views of the physician user's interface of Physician's Mobile App portal screen device, displaying the Doorbell Doc logos 902 and 918, menu selection treatment options, including Urgent Care 904, My Treatment Plan 906, My Medical Records 908, 911 dispatch 910, My Prescriptions 912, Doctor's Location 914. System 900 illustrates by showing the user interface of the physician's mobile app screen displaying 916 and Doorbell Doc logo 918, as well as querying of the patient regarding the need for scheduling in-person hands on examination by a nearby physician 920 and the selection for the anatomical region of symptomatic concern 922.

System 900 illustrates a physician's mobile app screen displaying a digitally modifiable 3-D full scale human avatar pictorial image in the likeness of the patient user 928 side by side views of the front view 924 rear view 926. System 900 illustrates a method 924 and 926, for our purposes the digitally modifiable 3D full-scale human pictorial image involves using software to create a three-dimensional model of the physician user, a digitally modifiable 3D full scale image in the likeness of the patient user.

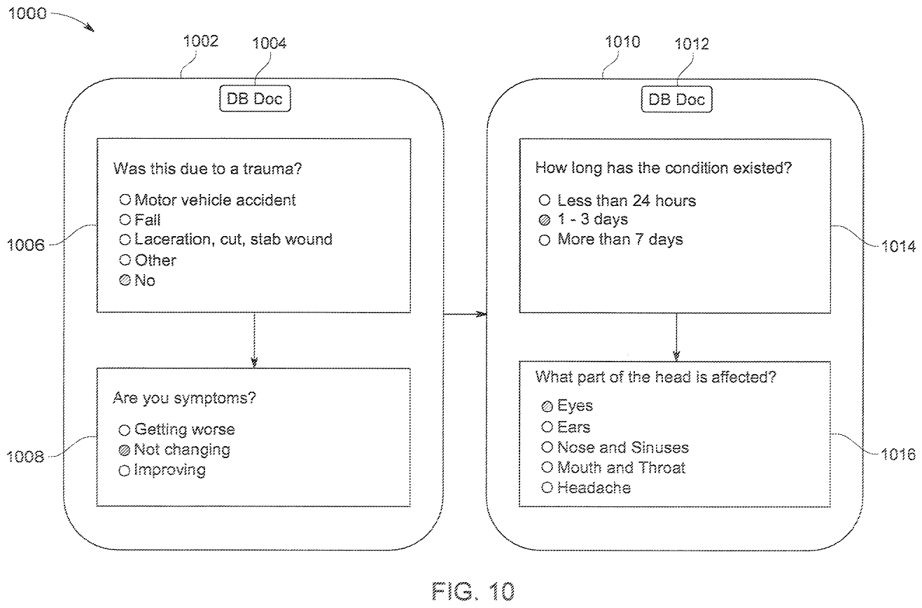

FIG. 10 system 1000 is like FIG. 7, is a method platform utilizing the patient's mobile device instrument for seeking non-traumatic medical treatment 1006, recording pertinent non-changing medical symptoms 1008 and history of illness that the patient is seeking in-person hands on treatment by a nearby physician. This method illustrates a condition of non-trauma for complaints of eye symptoms lasting ongoing for 1-3 days, 1014 reported by the patient user from the patient's mobile portal device and seeking in-person hands on examination by a nearby physician. System 1000 shows patient user interface querying the anatomical location of symptomatic complaints 1016.

System 1000 shows the user interface 1002 and 1010 a method platform utilizing the patient's mobile device instrument for seeking non-traumatic medical treatment, recording pertinent non-emergency medical symptoms and history of illness that the patient is seeking in-person hands on treatment by a nearby physician.

System 1000 shows the user interface of the patient's mobile app screen the Doorbell Doc app logo 1004 and 1012.

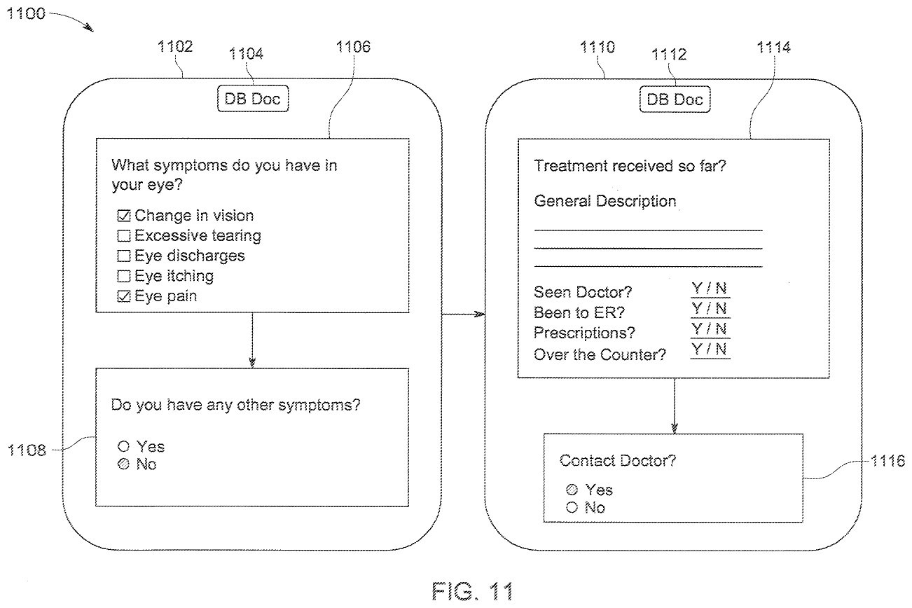

FIG. 11 system 1100 shows a user interface from the patient's mobile app 1102 and 1110 continuing from FIG. 10, system 1000 is a method platform utilizing the patient's mobile device instrument for seeking non-traumatic medical treatment, recording pertinent non-changing medical symptoms and history of illness that the patient is seeking in-person hands on treatment by a nearby physician. This method illustrates the App portal algorithmically programmed queries to precisely capture the patient's users symptomatic eye complaints 1106 and possible other symptomatic complaints 1108 and history of previous and or ongoing treatments received for this condition 1114 and the option to schedule an appointment for in-person hands on examination by a nearby physician 1116. System 1100 shows the user interface of the patient's mobile app screen the Doorbell Doc app logo 1104 and 1112.

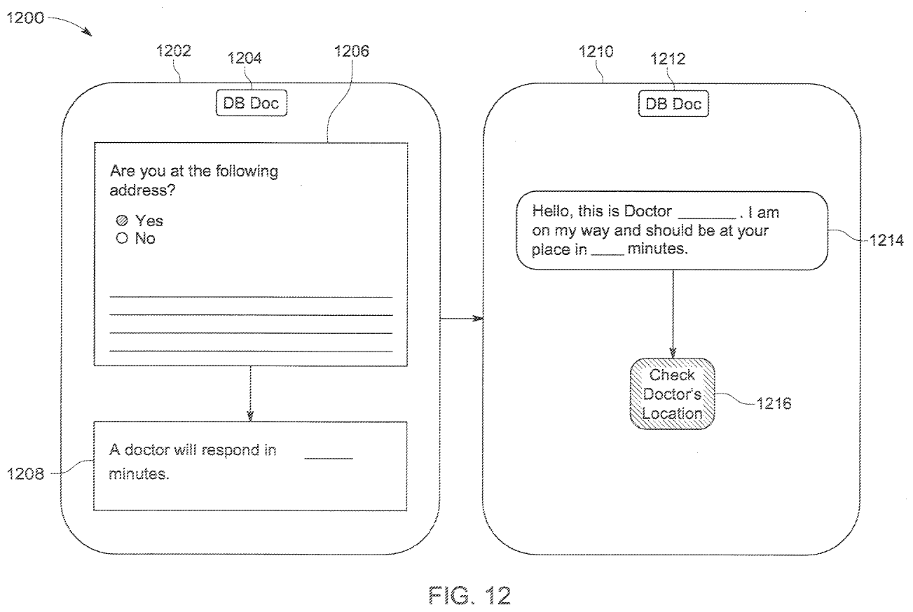

FIG. 12 system 1200 shows a user interface from the patient's mobile app 1202 and 1210 continuing from system 1100 FIG. 11, system 1200 is a method platform utilizing the patient's mobile device instrument for seeking non-traumatic medical treatment, confirming address and location to receive an in-person hands on examination by a nearby physician 1206. System 1200 shows the user interface of the patient's mobile app screen the Doorbell Doc app logo 1204 and 1212.

System 1200 shows user interface from patient's mobile app confirming the patient's user's exact present location to receive in-person hands on examination by a nearby physician 1206, the physician estimated arrival time to perform their in-person hands on examination and the nearby physician's response to the patient seeking in-person hands on treatment and tracking the physician's location and time of arrival to the patient's location for medical treatment 1208. System 1200 shows the user interface from the patient's mobile app with the on-screen display of the nearby physician's introduction 1214 to the patient confirming the scheduled in-person hands on examination by a nearby physician. Patients can track on their patient mobile device screen the exact location and time of the arrival of the nearby physician 1216.

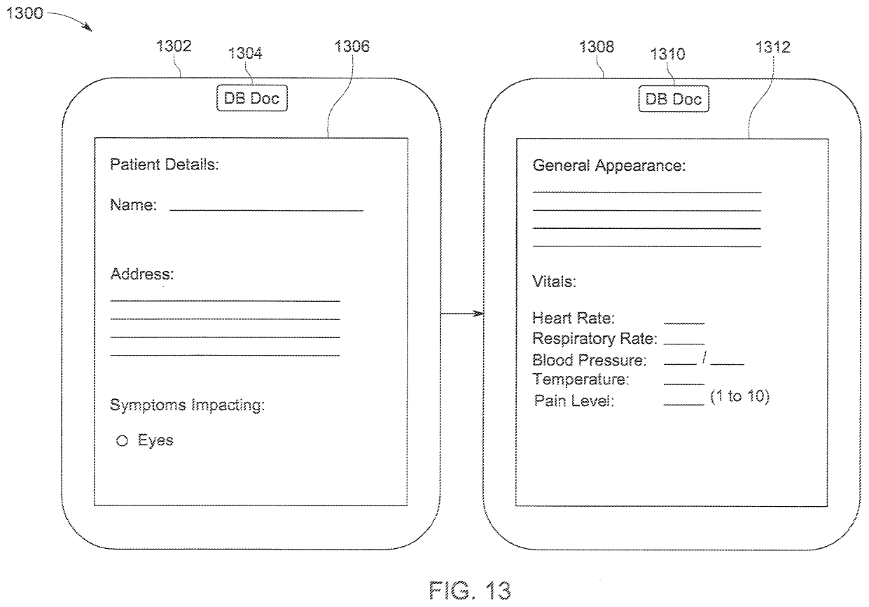

FIG. 13 system 1300 shows a physician's user interface on the physician's mobile app device screen, displaying the patient's demographics. For our purposes here, the embodiments of the illustration are not complete but suggestive of the scope of information viewed by the nearby physician on the physician mobile app device screen, including but not limited to the patient's name, current address location to receive in-person treatment, self-reported symptomatic complaints described by the patient generated by the programmed medical algorithm questions that auto-transcribes a written medical history narrative, describing the patient's symptoms that correspond to the 11 human organ systems that are possible contributing factors to the patient's disease and diagnosis, along with their modifiable 3-D full scale human avatar in their likeness, illustrating the precise location of their symptomatic complaints and need for treatment by a nearby hands on physician 1306. This is a method illustrating the process for the physician user to utilize a patient's medical histories, the clinical examination findings to access the medical database system 130 for accurate diagnosis and treatment plans.

System 1300 shows the physician user's interface on the physician's mobile portal app screen device 1302 and 1308, along with the Doorbell Doc logos 1304 and 1310. System 1300 shows the user interface on the physician's mobile app device, the first page of the examination template programmed by the digital medical algorithm, displaying the area to record human vital signs 1312.

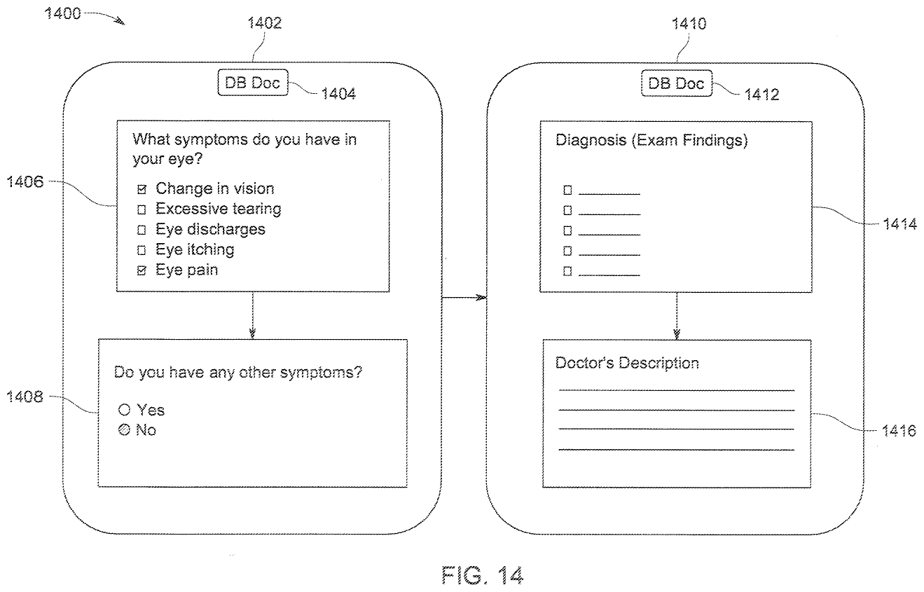

FIG. 14 system 1400 shows a user interface from the physician's mobile app screen 1402 and Doorbell Doc logo displayed on physician interface screen 1404. FIG. 14, system 1400 is a method platform utilizing the physician's mobile device instrument for viewing and analyzing the patient's request for seeking non-traumatic medical treatment, recording pertinent non-changing medical symptoms and history of illness that the patient who is seeking in-person hands on treatment by a nearby physician. This method illustrates the App portal algorithmically programmed queries to precisely capture the patient's users symptomatic eye complaints 1406 and possible other symptomatic complaints 1408. System 1400 further illustrates a physician's mobile app screen display 1410 and Doorbell Doc's logo 1412. This display also illustrates the in-person hands on physician's examination findings, diagnosis, and description of clinical findings.

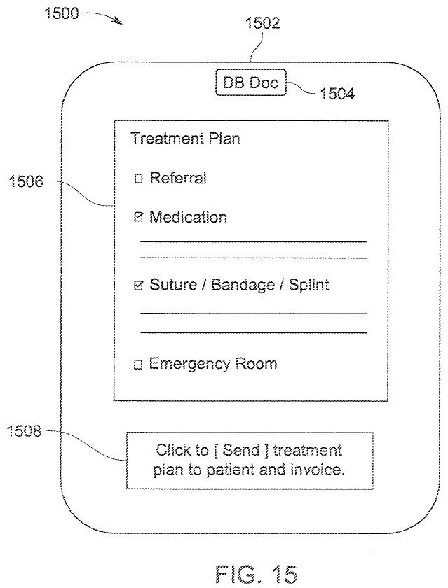

FIG. 15 system 1500 shows a user interface from the physician's mobile app screen 1502 and Doorbell Doc logo displayed on physician interface screen 1504. FIG. 15, system 1500 is a method platform utilizing the physician's mobile device instrument for documenting medical treatment 1506 utilizing the library of clinical diagnosis (not shown) and treatments generated from the 11 human organ systems (not shown) that are potentially responsible for the clinical diagnosis and most accurate treatment plan. Step 1508 illustrates the in-person hands on examining uploading the physician's clinical data record into the patient's electronic health record system, via the mobile app system platform.

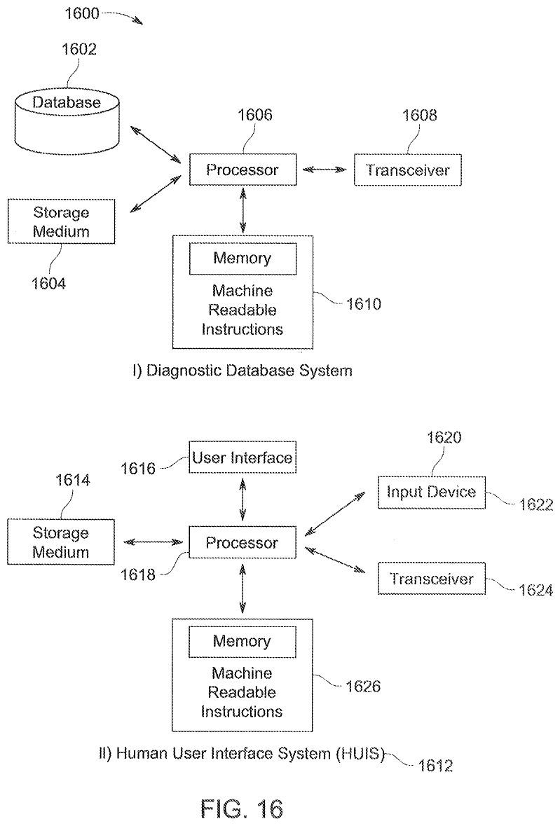

FIG. 16 illustrates an integrative diagnostic database system platform, system 1600. System 1600 provides database 1602 that is in communication with a processor 1606, that is in communication with a storage medium 1604, and a plurality of modules. One such module is a transceiver 1608. The transceiver 1608 interfaces with a communication network such as a mobile telephony network, a TCP/IP network, or circuit-switched network. System 1600, via transceiver 1608, is in communication with one or more diagnostic client devices (not shown) over the communication network.

System 1600 further provides a memory 1610 in communication with a processor 1606. Machine readable instructions 1610 are resident in memory 1610 for execution by the processor 1606 to cause processor 1606 to carry out the steps of methods described herein. For purposes of the present disclosure, the term “machine-readable medium” refers to any tangible or non-transitory medium that can store, encoding or carrying instructions for execution by the machine and that cause the machine to perform any one or more of the methodologies of the present disclosure, or that can store, encoding or carrying data structures utilized by or associated with such instructions. The term “machine readable medium” includes, but is limited to, solid-state memories, and optical and magnetic media. Specific examples of machine-readable media include non-volatile memory, including by way of example, semiconductor memory devices, e.g., EPROM, EEPROM, and flash memory devices; magnetic disks such as internal hard disks and removable disks; magneto-optical disks; and CD-ROM and DVD-ROM disks. The term “machine-readable medium” may include a single medium or multiple media (e.g., a centralized or distributed database, and/or associated caches and servers) that store the one or more instructions or data structures. Memory 1610 may be implemented in a random-access memory (RAM), a hard drive, a read only memory (ROM) or a combination thereof. One of the components of memory is program module (not shown) 125. A program module contains instructions for controlling processor 1606 to execute functional methods. Instructions 1610 are stored on a storage medium 1604 for loading into memory 1610. Storage medium 1604 may be physically distant to the processor 1606, local to the processor 1606, or some combination of local and distant.

Processor 1606 is further in communication with database 1602 for storing a record (not shown), e.g., record 140. It is to be understood that multiple records may be stored in database 1602. The record may include data of record 140 (not shown). For purposes of the present disclosure, the term “processor” refers to a device that performs the basic operations in a computer. A microprocessor is one example of a processor 1606, Program Module (not shown), For the purposes of the present disclosure, the term “database” or “data record” refers to a structured collection of records or data that is stored in a computer system. The structure is achieved by organizing the data according to a database model. The model in most common use today is the relational model. Other models such as the hierarchical model and the network model use a more explicit representation of relationships (see below for explanation of the various database models). A computer database relies upon software to organize the storage of data. This software is known as a database management system (DBMS). Database management systems are categorized according to the database model that they support. The model tends to determine the query languages that are available to access the database. A great deal of the internal engineering of a DBMS, however, is independent of the data model, and is concerned with managing factors such as performance, concurrency, integrity, and recovery from hardware failures. In these areas there are large differences between products, Database 1602, system Database. For the purposes of the present disclosure, the term “data storage medium” or “data storage device” refers to any medium or media on which data may be stored for use by a computer system. Examples of data storage media include floppy disks, Zip™ disks, CD ROM, CD-R, CD-RW, DVD, DVD-R, memory sticks, flash memory, hard disks, solid state disks, optical disks, etc. Two or more data storage media acting similarly to a single data storage medium may be referred to as a “data storage medium” for the purposes of the present disclosure. A data storage medium may be part of a computer. For the purposes of the present disclosure, the term “data storage medium” or “data storage device” refers to any medium or media on which data may be stored for use by a computer system. Examples of data storage media include floppy disks, Zip™ disks, CD ROM, CD-R, CD-RW, DVD, DVD-R, memory sticks, flash memory, hard disks, solid state disks, optical disks, etc. Two or more data storage media acting similarly to a single data storage medium may be referred to as a “data storage medium” for the purposes of the present disclosure. A data storage medium may be part of a computer.

Storage Medium 1604 creates a record 140 (not shown) including clinical data, health history with an expanding library of descriptive terms used by the patient user for documented symptoms, medications, modifiable avatar, the 11 human organ systems correlating with symptomatic complaints, clinical findings, diagnostic data, and treatment plans, medications, medications allergies. For example, record 140 (not shown) is within database 1602 and may include physician data such as treatment data, and location data. Location data is a location of a physician of physician's office and includes a latitude/longitude (lat/Lon) coordinate. In this fashion, a potential patient may access the record in database 1602 via transceiver 1608 and processor 1606 to obtain the location of the physician of nearby physician or nearby physician's offices. This assists the potential patient to locate local potential nearby available treating physicians. Alternatively, a potential patient, via client device 102 (not shown), may access an in-person treating physician based on the nearby physician's availability.

System 1600 further provides 1612 human user interface system which illustrates the function of diagnostic database system, including a storage medium 1614, a module such as a user interface 1616 provides an input that is a man-machine interface (MMI) with controls for a user to command the system 1600. User interface 1616 further provides an output (not shown) for communication prompts, alerts, menus, dialogs, and other signals that are perceptible to the user. In a preferred embodiment, user interface 1616 supports a tactile and graphical user environment.

System 1600 further provides a memory 1626 that is in communication with a processor 1618. Machine-readable instructions 1626 are resident in memory (1626) for execution by processor 1618. Instructions 1626 are stored on a storage medium 1614 for loading into a memory 1626 to cause processor 1618 to carry out the steps of methods described herein. Storage medium 1614 in turn may be physically distant to processor 1618, local to processor 1618, or some combination of local and distant. Processor 1618 is also in communication with an input that inputs data such as patient data and physician data. The input data includes, but is not limited to a scale, a sphygmomanometer, a spirometer, and a 3D digitally modifiable avatar in the likeness of the patient user. It is within the contemplation of the present disclosure that input device 1620 or 1622 may also be any suitable input device capable of assaying a patient and machine-readable data. Transceiver 1624 is a module that is an interface to a communication network such as a mobile telephony network, a TCIP/IP network, a circuit-switched network, or the like.

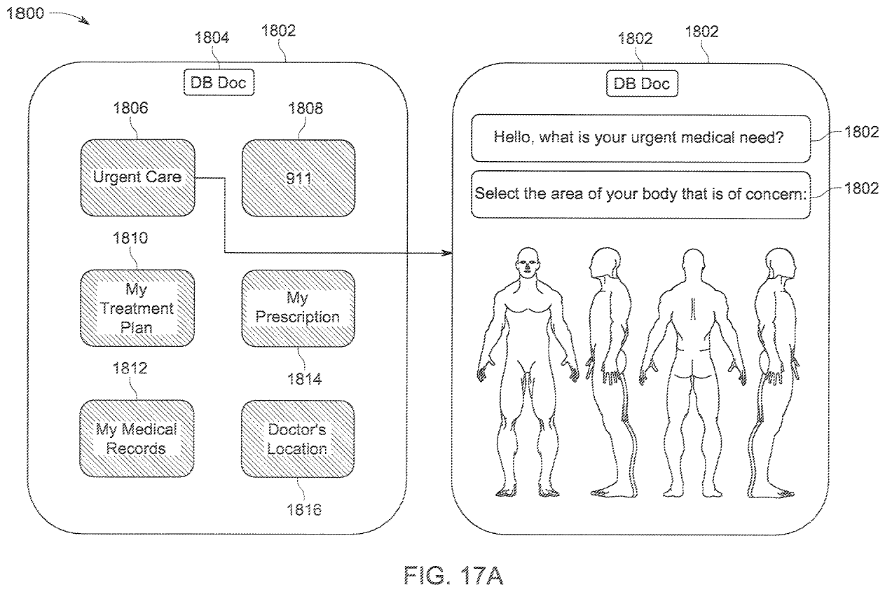

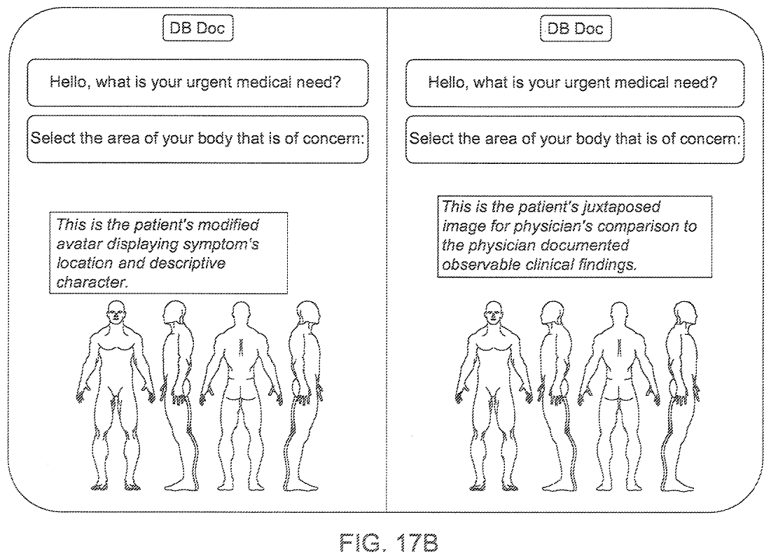

FIG. 17A system 1800 for our illustrative example shows the Physician user's interface from the physician's mobile app device portal screen 1802, displaying the patient user's queried responses involving their need for urgent medical treatment along with the two juxtaposed modifiable 3-D full scale human avatar digital images in the likeness of the patient user, appearing on the physician user's mobile screen device. This allows the in-person examining physician to view and compare the patient user's digitally illustrated symptoms using the modifiable 3-D avatar and the physician user's documented clinical findings on the juxtaposed modifiable 3-D avatar image in the likeness of the patient user.

FIG. 17B system 1800 provides further explanation of an example of the physician user's displayed view the modifiable 3-D full scale human avatar in the likeness of the patient user, viewing on the Physician mobile screen device the patient user's modified selection of symptomatic complaints using their 3-D avatar digital image to illustrate the anatomical locations of symptomatic complaints and the physician user's documentation of their in-person hands clinical examination findings.

What is claimed is:

1. A method for self-reporting patient medical symptoms to a physician using at least one digitally interactive, three-dimensional, full-sale, human body image modifiable anatomical avatar to be depicted in a patient's image, said method comprising:
(A) transmitting a patient mobile application to a patient digital mobile device for a patient experiencing medical symptoms and seeking medical treatment, said patient mobile application comprising said modifiable anatomical avatar and a plurality of differential diagnostic questions to assess, and digitally self-report, a patient's symptoms to a physician;
(B) transmitting a physician mobile application to a physician digital mobile device for a physician who is available to examine patients within a designated proximity to a physician, said physician mobile application comprising a network and plurality of digital platforms to allow a physician's access to a patient's digital healthcare records and demographic information, which facilitates the provision of patient healthcare treatment strategies;
(C) receiving from said patient mobile application said digital healthcare records comprising a patient's answers to said plurality of differential diagnostic questions paired with at least one of said modifiable anatomical avatars, modified to depict a patient's symptoms, wherein said digital healthcare records are stored in a database, which is accessible via a network, by a patient and at least one physician;
(D) receiving from said patient mobile application an opt-in request to locate a nearby physician and facilitate an in-person appointment scheduling request;
(E) receiving from said patient mobile application an approval to participate in, and pay for, an in-person medical examination according to said in-person appointment scheduling request
(F) transmitting said in-person appointment scheduling request to said physician mobile application via said network;
(G) receiving an acceptance of said in-person appointment scheduling request from said physician mobile application;
(H) utilizing, in combination, said physician mobile application, said digital healthcare records stored in said database and accessible via said network, to complete a patient's in-person medical examination; and
(I) utilizing said physician mobile application, said digital healthcare records and data obtained during an in-person patient medical examination to prescribe a customized patient treatment plan, which is added to said digital healthcare records and stored in said database.

2. The method of claim 1 wherein a patient can view said digital healthcare records comprising said modifiable anatomical avatar through said patient mobile application, and a corresponding physician can view said digital healthcare records comprising said modifiable anatomical avatar through said physician mobile application, allowing a physician digital access to said same modifiable anatomical avatar as modified by a patient, in order to illustrate a patient's self-reported medical symptoms, and to illustrate a physician's corresponding confirmed clinical findings.

3. The method of claim 1 wherein said plurality of digital platforms comprises a system triage platform, providing rapid physician analysis based on a patient's self-reported symptoms and said digital healthcare records submitted via said patient mobile application and a corresponding physician's clinical findings and treatments submitted via said physician mobile application.

4. The method of claim 1 wherein said plurality of digital platforms comprise an emergency response platform, allowing a patient with emergency medical needs to receive a prompt via said patient mobile application to dial an emergency services response number such as 911 via said patient mobile application to request emergency services at a patient's current location.

5. The method of claim 1 wherein said plurality of digital platforms comprise a non-emergency platform, allowing a patient with non-emergency medical needs to request medical services via said patient mobile application from a physician via said physician mobile application.

6. The method of claim 1, wherein said patient mobile application is integrated with said digital healthcare records, comprising all captured healthcare information from a patient's self-reported symptoms, said modifiable anatomical avatar as modified by a patient in order to illustrate a patient's self-reported medical symptoms, and a physician's recorded clinical findings and treatments, wherein said digital healthcare records are formatted to be stored in and retrieved from said database via said network, permitting a patient to access said digital healthcare records and share said digital healthcare records with any medical providers or healthcare agencies of a patient's choice.

7. The method of claim 1, wherein said modifiable anatomical avatar comprises a modifiable virtual matrix, which produces illustrations of anatomical regions causing a patient's symptoms, records an examining physician's clinical findings of the entire human organs system, allows both a patient and a physician to locate a patient's symptoms and severity of physical disease or injury, provides appropriate timely medical treatment needed to help cure a patient's illness, and facilitates providing immediate comprehensive medical diagnoses, assessments and treatments, thereby establishing a consistent protocol in healthcare access and delivery.

8. The method of claim 1, wherein a patient's self-reported symptoms are systematically correlated with each relevant human organ system via said modifiable anatomical avatar, which improves the measurable physiological impact on the respective human organ system, facilitates capturing and monitoring multiple organ systems simultaneously, and improves prognosis for recovery.

9. A system for self-reporting patient medical symptoms to a physician using at least one digitally interactive, three-dimensional, full-sale, human body image modifiable anatomical avatar to be depicted in a patient's image, said system comprising:
(A) a patient mobile application;
(B) physician mobile application;
(C) a network;
(D) a database comprising digital healthcare records;
(E) a storage medium;
(F) a memory module;
(G) a program module;
(H) a processor; and
(I) a plurality of digital platforms providing integrated functionality within said system.
